(19) United States
(12) Patent Application Publication
(10) Pub. No.: US 2023/0405062 A1
(43) Pub. Date: Dec. 21, 2023

(54) GOS PRE-CONDITIONING L. REUTERI AND GOS IN FINAL FORMULATION

(71) Applicant: BIOGAIA AB, Stockholm (SE)

(72) Inventors: Stefan ROOS, Uppsala (SE); Marie Noëlle HORCAJADA, ECHENEVEX (FR); Nicolas BONNET, NANGY (FR); Magalie SABATIER, LAUSANNE 26 (CH); Bertrand BOURQUI, BERN (CH); Guénolée Eliane Marie PRIOULT, BERN (CH); Florac DE BRUYN, LAUSANNE (BE)

(21) Appl. No.: 18/002,184

(22) PCT Filed: Mar. 11, 2022
(86) PCT No.: PCT/SE2022/050245

§ 371 Date: Dec. 16, 2022

(30) Foreign Application Priority Data
Mar. 12, 2021 (EP) 21162450.7

(51) Int. Cl. A61K 35/747; A23L 33/135; A23L 33/125; C12N 1/20; A61K 47/26; A61P 19/00; A61P 3/02

(52) U.S. Cl. CPC A61K 35/747; A23L 33/135; A23L 33/125; C12N 1/205; A61K 47/26; A61P 19/00; A61P 3/02; C12R 2001/225

(57) ABSTRACT

The invention herein relates generally to enhancing the survival and activity of probiotic Lactobacillus reuteri strains in mammals by pre-conditioning L. reuteri with GOS during cultivation and to a surprisingly high symbiotic effect of such a pre-conditioned probiotic L. reuteri strain when administered together with GOS further added in the composition. The invention therefore comprises methods for cultivating and manufacturing probiotic L. reuteri strains, and preparing products containing such pre-conditioned strains in combination with additional amounts of GOS. In more detail the present invention relates to a probiotic composition comprising a probiotic L. reuteri strain, obtained by a step of growing the bacteria in a medium comprising GOS, and administering the pre-conditioned bacteria together with further GOS in the final product. The inventors have found that such method induces surprisingly high and unexpected symbiotic beneficial effects of the probiotic bacteria in the gastrointestinal tract. The probiotic composition also induces an increased calcium and iron solubility.

### TECHNICAL FIELD

The invention herein relates generally to enhancing the survival and activity of probiotic Lactobacillus reuteri strains in mammals by pre-conditioning L. reuteri with galacto-oligosaccharides (GOS) during cultivation and to a surprisingly high synbiotic or synergistic effect of such a pre-conditioned probiotic L. reuteri strain when administered together with GOS further added in the composition. The invention therefore comprises methods for preparing a probiotic composition, and a probiotic composition comprising a probiotic L. reuteri strain pre-conditioned with GOS and comprising additional GOS, and uses thereof. The inventors have found that such a method and composition achieve surprisingly high and unexpected synbiotic beneficial effects in the gastrointestinal tract. The composition also induces an increased iron and calcium solubility.

### BACKGROUND

Probiotics are defined as “live microorganisms that, when administered in adequate amounts, confer a health benefit on the host.” This is the widely accepted scientific definition around the world (see e.g., Hill et al. 2014; Nat. Rev. Gastro. & Hepathology, 11: 506-514). Probiotic products (which are usually dietary supplements or foods) may be recommended for different conditions or symptoms an individual is experiencing. Lactic acid producing bacteria, such as Lactobacilli, are commonly used as probiotics in various types of foods, for example yoghurt. Growth and colonization of harmful microorganisms can be prevented by such lactic acid producing bacteria through their own colonization inside the intestinal system, through competition of available nutrients and/or through production of specific substances, such as hydrogen peroxide, bacteriocins and/or organic acids, including lactic and acetic acid, that lowers the intestinal pH. It is well established that interactions between host and gut microbes are fundamental to health and well-being of the host. Intestinal microbiota generates metabolites that provide the host with nutrients but may also be involved in the immune response and in regulation and development of the host's immune system as well as reducing inflammation and preventing allergic responses.

Prebiotics are compounds that induce the growth or activity of beneficial microorganisms, such as bacteria and fungi, by selectively stimulating their growth and/or activity. Prebiotics are substrates that are selectively utilized by such host microorganisms conferring a health benefit (see e.g., Gibson et al. 2017; Nat. Rev. Gastro. & Hepathology, 14: 491-502). In the gastrointestinal tract, prebiotics can therefore alter the composition of the gut microbiome. Dietary prebiotics are typically nondigestible (see e.g., https://en.wikipedia.org/wiki/Dietary_fiber) fiber compounds that pass undigested through the upper part of the gastrointestinal tract and stimulate the growth or activity of advantageous bacteria that colonize the large bowel by acting as their substrate. Various compounds have been tested to determine their function as prebiotics. Fructo-oligosaccharides (FOS), galacto-oligosaccharides (GOS), and trans-galacto-oligosaccharides (TOS) are the most common prebiotics. Consuming certain prebiotics can, thus, improve immunity functions by increasing the population of microorganisms associated with human health, and animal and human studies have shown that prebiotics can decrease the population of harmful bacteria. Prebiotics are, thus, ingredients, providing health benefits when fermented by the native microflora in the intestine of a subject or by probiotic bacteria ingested simultaneously with the prebiotics.

Combining particular types of prebiotics and probiotics is generally known to give rise to certain synergistic effects. In particular, the effect of combined prebiotic GOS and probiotic microorganisms, such as Lactobacillus and Bifidobacteria, has been documented (see e.g. YUTAKA KANAMORI, MD et al., Digestive Diseases and Sciences, Vol. 46, No. 9 (September 2001), pp. 2010-2016 (©2001)). It has been demonstrated that the synbiotic effect between prebiotics and probiotics is due to the consumption of the prebiotic by the probiotic within the gastrointestinal (GI) tract of the subject. The manufacturing procedure of probiotic lactic acid producing bacteria is typically standardized and involves a step of fermenting the bacteria in a growth medium comprising a carbohydrate source, such as a sugar, for example glucose, fructose, sucrose, lactose or dextrose. Following the fermentation, the probiotic bacteria are usually protected and frozen or freeze-dried and packaged into a finished product.

The ingestion of probiotic lactic acid bacteria has various effects in vivo. One such important effect is the bio-accessibility of essential minerals, which can be significantly influenced by the presence of such probiotic lactic acid bacteria. Although the content of minerals in the body depends primarily on their supply from food intake and they are absorbed mainly in the GI tract, mere provision of minerals via food intake does not necessarily lead to a sufficient mineral bio-accessibility. The main factors affecting mineral bio-accessibility are the content of minerals in the food, synergistic and antagonistic interactions between minerals in the food and in the GI tract, the presence of complexing or chelating compounds in the food, and the health state of the organism and its age. Also, the intestinal microflora, probiotics and prebiotics significantly influence the bio-accessibility of minerals and can thereby increase or decrease the absorption of such minerals.

Minerals are essential for all living species, even though the specific requirements differ between species. Minerals have a great number of important functions in the human organism and include e.g., iron and calcium, among many others. Mineral deficiencies, i.e., low or suboptimal amounts of such minerals in the body, typically by ingesting minerals below recommended RDA amounts, can lead to diseases and so can mineral excess. It is therefore pivotal to obtain the correct amounts of minerals at the correct ratios for optimal health. Most natural diets will provide these minerals in appropriate balances, but there are situations when this is not enough to maintain health.

Recent studies have produced new information on microbiota—mineral interactions and have mainly concerned the impact of intestinal bacteria on the metabolism of calcium and iron. Given the aging population and the rising prevalence of osteoporosis among postmenopausal women and men, the new data on calcium metabolism and hence on bone metabolism and its improvement are crucial to the development of the field. In contrast, the results of research into adverse interactions between iron and the intestinal microbiota show that there is a crucial need for further studies on the bio-accessibility of this mineral in order to prepare new recommendations regarding iron supplementation.

Also, a common problem with oral administration of probiotic bacteria is that they arrive at their destined location in the gastrointestinal tract in insufficient amounts and/or that the activity of the probiotic bacteria in these locations of the intestinal tract, where they assert their effects, is inadequate. The survival, viability and engraftment of the probiotic bacteria in the gastrointestinal tract is thus a significant challenge for those who manufacture probiotic products. As a consequence, the dosage of probiotic bacteria has to be increased and/or more frequent administration is required to obtain the desired probiotic effect. This further leads to problems with unnecessary costs, undesirable frequency of intake and possibly also decreased health benefits when the probiotic bacteria do not sufficiently provide the desired effects. Thus, it is desired to improve the probiotic effect without increasing the dosage.

### SUMMARY OF THE INVENTION

It is an object of the current invention to provide more efficient means, methods and therapies of improving the survival and probiotic effects of probiotic microorganisms, such as lactobacilli, when administering such probiotics to a subject or a patient in need thereof. Moreover, it is a particular object to prevent and/or treat mineral deficiency in a subject or in a patient in need thereof, particularly mineral deficiencies of minerals, e.g., selected from iron and/or calcium.

These objects are solved by the current invention as addressed below.

The present invention is defined below and in the independent claims. Further embodiments of the invention are defined in the specification and in the dependent claims.

In a first aspect, the invention provides a method for preparing a probiotic composition. The method comprises the steps:

a) pre-conditioning of a probiotic Lactobacillus reuteri (L. reuteri) strain by:
ai) cultivating a probiotic L. reuteri strain in the presence of galacto-oligosaccharide (GOS) in a growth medium, thereby pre-conditioning the probiotic L. reuteri strain; and
aii) harvesting the pre-conditioned probiotic L. reuteri strain from the growth medium;

b) adding GOS to the pre-conditioned probiotic L. reuteri strain obtained from step aii) to prepare a probiotic composition.

The probiotic composition prepared by the inventive method comprises the pre-conditioned probiotic L. reuteri strain and the supplemented or added GOS.

In step ai) of the inventive method, pre-conditioning of a probiotic L. reuteri strain occurs by cultivating or culturing a probiotic L. reuteri strain in the presence of GOS in a growth medium, thereby pre-conditioning the probiotic L. reuteri strain. Cultivation of a probiotic L. reuteri strain in the presence of GOS leads to formation of the pre-conditioned probiotic L. reuteri strain.

According to a second aspect, the invention provides a probiotic composition comprising an effective amount of a pre-conditioned probiotic L. reuteri strain, and additionally an effective amount of GOS. The pre-conditioned probiotic L. reuteri strain has been prepared by cultivating a L. reuteri strain in the presence of GOS in a growth medium.

In a third aspect, the invention provides a non-therapeutic use of the inventive probiotic composition comprising an effective amount of a pre-conditioned probiotic L. reuteri strain supplemented with an effective amount of GOS, the probiotic composition is obtainable by the inventive method or as described herein, for increasing or boosting the probiotic effect, e.g., of the probiotic L. reuteri strain in the digestive tract of a healthy subject. The increase of the probiotic effect may be manifested e.g., in increasing mineral bio-accessibility in the intestinal tract of the healthy subject, promotion of a healthy microbiota in the intestinal tract of the healthy subject, promotion of the survival of the L. reuteri in the colon and thus a promotion of the health benefits associated to the L. reuteri strain, increasing metabolic activity of the gut microbiota and/or increasing bone formation and/or bone mineralization and/or increasing maturation and/or activity of osteoblasts in the healthy subject.

According to a fourth aspect, the invention provides a probiotic composition comprising an effective amount of a pre-conditioned probiotic L. reuteri strain and an effective amount of GOS obtainable by the inventive method or as described herein, for use as a medicament.

According to further aspects, the invention provides a probiotic composition comprising an effective amount of a pre-conditioned probiotic L. reuteri strain and an effective amount of GOS obtainable by the inventive method for use in the prevention and/or treatment of mineral deficiency in a subject in need thereof, a digestive dysfunction, a dysbiosis, bone loss or for use in promoting growth and/or development of bone or teeth in children.

Further preferred embodiments are described herein below and in the dependent claims.

### BRIEF DESCRIPTION OF THE DRAWINGS

FIG. 1: shows the short SHIME colonic in vitro model used to assess the impact of probiotic strains on microbiota metabolism and engraftment of probiotic strains.

FIG. 2: shows calcium solubility measured at 6 h and 24 h in the colon. As can be seen in FIG. 2, pre-conditioned probiotic L. reuteri strain boosts calcium solubility in colon at both 6 h and 24 h in presence of GOS. This suggests that a pre-conditioned L. reuteri strain combined with GOS can be used to promote calcium absorption in a subject in need thereof.

FIG. 3: shows the improved iron solubility (bio-accessibility) in the colon at 48 h. GOS and pre-conditioned L. reuteri boost iron solubility (bio-accessibility) in the colon suggesting that the combination of both GOS and pre-conditioned L. reuteri can promote iron absorption in a subject in need thereof.

FIG. 4A: shows that survival of L. reuteri DSM17938 and Streptococcus thermophilus NCC2496 in presence or absence of GOS in the colon after 48 h incubation, particularly a positive effect of GOS on L. reuteri survival and negative effect of GOS on Streptococcus thermophilus survival.

FIG. 4B: shows that the survival of pre-conditioned L. reuteri DSM17938 is better than non-pre-conditioned L. reuteri strain in presence of GOS

FIG. 5: shows the lactic acid production (difference between the level at 48 h and 6 h). GOS alone induces a dramatic increase of lactic acid production in the colon. Addition of L. reuteri in addition to GOS further enhances the lactic acid production. Pre-conditioned L. reuteri in combination of GOS boosts even more than L. reuteri (not pre-conditioned) the production of lactic acid in the colon showing an overall increase of the metabolic activity of the microbiota. Lactic acid-producing bacteria are more active in the colon when GOS and pre-conditioned L. reuteri are added for 48 h.

FIG. 6: shows the pH decrease in the colon (difference between 48 h and 6 h) (right panel) and acetic acid production (difference between 48 h and 6 h) (left panel). GOS alone induces a pH drop and higher acetic acid production. L. reuteri added on top of GOS in the colon amplifies further the effect on pH and acetic acid production, meaning pH drops even more when L. reuteri is added to GOS. Pre-conditioned L. reuteri combined with GOS induces a stronger pH decrease in the colon. Higher level of acetic acid is also produced in presence of pre-conditioned L. reuteri and GOS, compared to GOS alone and GOS plus L. reuteri. pH is a marker of acid production. Both lactic acid and acetic acid are strongly induced by pre-conditioned L. reuteri in presence of GOS.

FIG. 7: shows early differentiation of osteoblasts at Day 7, measuring alkaline phosphatase (ALP) activity. The results show that GOS+pre-conditioning of a L. reuteri strain led to increased ALP activity in osteoblasts, suggesting that the pre-conditioned L. reuteri strain+GOS can stimulate osteoblast differentiation.

FIG. 8: illustrates alizarin red absorption, a marker of mineral matrix that is synthetized by differentiated osteoblasts. Results show that alizarin red level is enhanced by the GOS+pre-conditioned L. reuteri strain compared to GOS +L. reuteri strain (without pre-conditioning), at day 21. This suggests that pre-conditioned L. reuteri strain+GOS can stimulate bone mineralization.

FIG. 9: illustrates MC3T3 migration speed, a measure of cell mobility, at day 4. Results show that cell migration is more significantly decreased by GOS+pre-conditioned L. reuteri compared to GOS alone or combined with L. reuteri strain (without pre-conditioning). This suggests superiority of pre-conditioned L. reuteri strain+GOS to stimulate osteoblast differentiation.

FIG. 10: illustrates MC3T3 Atf4 gene expression, a marker of early osteoblast differentiation, at day 4. Results show that both SCFAs blends from GOS+L. reuteri or GOS+pre-conditioned L. reuteri conditions increased significantly the mRNA expression of Atf4 vs control condition.

FIG. 11: illustrates MC3T3 Ocn gene expression, a marker of osteoblast mineralization, at day 4. Results indicates a superiority of preconditioned L. reuteri+GOS to stimulate of osteoblast mineralization vs GOS+L. reuteri (without preconditioning), through respective SCFAs blends.

### DETAILED DESCRIPTION

The invention herein provides a way of enhancing probiotic effects of Lactobacillus reuteri strains in a mammal, using specific substrate components during fermentation when manufacturing the probiotic L. reuteri strains and further providing a surprisingly high synbiotic or synergistic effect of the combination of the pre-conditioned probiotic L. reuteri strain and galacto-oligosaccharides (GOS) as further added to produce the final probiotic composition. The inventors of the present invention have, thus, developed a method, which comprises the use of GOS during cultivation of the probiotic L. reuteri strain, so called “pre-conditioning” with GOS, and addition of additional or supplemental GOS to produce the final probiotic composition. This method surprisingly results in an unexpectedly high probiotic effect of the L. reuteri strain, particularly a significant increase of survival of the probiotic L. reuteri strain, an increased bone formation and/or bone mineralization, an increased metabolic activity of the microbiota, increased bio-accessibility of minerals, e.g., by increasing mineral solubility, accessibility and/or absorption of minerals within the gastrointestinal tract of a subject or patient upon administering the pre-conditioned probiotic L. reuteri strain, as described herein. This beneficial and unexpected high effect is particularly generated due to the combined administration of additional GOS as a prebiotic and the (GOS-) pre-conditioned probiotic L. reuteri strain in one composition to a subject or patient in need thereof or in the form of a kit of parts.

The present invention therefore aims to provide probiotic compositions and uses, which, among others, increase mineral bio-accessibility and, hence, also mineral absorption in the gastrointestinal tract. In detail, such solutions are probiotic compositions comprising a GOS pre-conditioned Lactobacillus reuteri strain in combination with GOS further added to produce the final product to be administered to a subject, such as in need of increased mineral uptake. The increased mineral solubility, accessibility, and absorption takes place in the gastrointestinal tract, such as in the duodenum, the jejunum, the ileum and/or in the colon.

The present invention also aims to provide methods for preparing a probiotic composition comprising a pre-conditioned probiotic L. reuteri strain in combination with GOS in the probiotic composition such that increased mineral solubility and bio-accessibility and also mineral absorption in the gastrointestinal tract can be achieved by virtue of the combination of such pre-conditioned L. reuteri strains in combination with GOS further added to produce the probiotic composition.

Objectives of the invention therefore relate to a method for preparing a probiotic composition comprising such a pre-conditioned probiotic L. reuteri strain in combination with additional GOS in the probiotic composition and, to the non-therapeutic and therapeutic use of a pre-conditioned probiotic L. reuteri strain in combination with GOS to, among others, increase mineral solubility, accessibility and/or absorption in the gastrointestinal tract of a mammal.

The improved non-therapeutic and therapeutic effects obtained by the combined administration of (GOS-) pre-conditioned probiotic L. reuteri and additional GOS, makes it possible to decrease the dosage and/or the frequency of administration of the probiotic and prebiotics, such as GOS, or to increase the beneficial effects of the same doses of conventional probiotics and/or prebiotics, required to obtain the sought after health effects.

In view of the above, the following is disclosed.

### Methods for Preparing a Composition Comprising Pre-Conditioned Probiotic L. reuteri Bacterium and Additional GOS

According to the first aspect the invention provides a method for preparing a probiotic composition. The method comprises the steps:

a) pre-conditioning of a probiotic Lactobacillus reuteri strain by:
ai) cultivating or culturing a probiotic L. reuteri strain in the presence of galacto-oligosaccharide (GOS) in a growth medium, thereby preconditioning the probiotic L. reuteri strain; and
aii) harvesting the pre-conditioned probiotic L. reuteri strain from the growth medium; and

b) adding GOS to the pre-conditioned probiotic L. reuteri strain obtained from step aii) to prepare a composition, typically referred to as a probiotic composition.

The composition prepared according to the inventive method comprises the pre-conditioned probiotic L. reuteri strain and additional GOS. Thus, step b) of the method prepares a composition comprising the pre-conditioned probiotic L. reuteri strain obtained from step aii) and adding GOS to the composition.

In step ai) of the inventive method, pre-conditioning of a probiotic L. reuteri strain occurs by cultivating a probiotic L. reuteri strain in the presence of GOS in a growth medium, thereby pre-conditioning the probiotic L. reuteri strain. In the context of the current invention, the term “pre-conditioning” of the probiotic L. reuteri bacteria means growing, such as cultivating, culturing or fermenting, the probiotic Lactobacillus reuteri strain with GOS, i.e., in the presence of GOS. In the inventive context, the terms “cultivation”, “culturing” and “fermentation” are used interchangeably. Cultivation of a probiotic L. reuteri strain in the presence of GOS leads to formation of the pre-conditioned probiotic L. reuteri strain.

In step aii), the pre-conditioned probiotic L. reuteri strain is then harvested from the growth medium. The term “pre-conditioned L. reuteri” refers to a probiotic L. reuteri strain or bacteria of the pre-conditioned L. reuteri strain produced by the inventive method including a pre-conditioning step with GOS.

Furthermore, in step b) of the inventive method, the composition is formed by combining the pre-conditioned probiotic L. reuteri strain obtained from step aii) and further adding GOS to prepare or produce the final probiotic composition.

A L. reuteri strain as used throughout the current invention may generally be any L. reuteri strain, more preferably any commercially available L. reuteri strain. In this context, the terms “L. reuteri strain” and “L. reuteri bacteria” may be used synonymously. It is noted thereby that in the last decades DNA analysis tools have become more sophisticated, which has enabled scientists to discover many new bacterial species as well as realizing that the species historically grouped under the Lactobacillus genus were too diverse and did not conform to nomenclature conventions. To keep the probiotic groups accurate and organized, the genus Lactobacillus was therefore split into 25 different genera, including 23 novel genera. As a result, many probiotics have recently been given new genus names. Therefore, an alternative genus name for Lactobacillus reuteri (L. reuteri) is Limosilactobacillus reuteri. Since the reclassification process was very recent, we will use the former genera nomenclature, i.e., Lactobacillus for clarity purposes in this application. The revised genera nomenclature and alternative names should, however, be readily obtainable by anyone using available reclassification tools.

Currently preferred bacteria provided in the compositions are bacteria of at least one L. reuteri strain or of multiple, i.e., at least two, L. reuteri strains, preferably as mentioned herein.

In a preferred embodiment of the current invention, the probiotic L. reuteri strain is at least one L. reuteri strain selected from the group consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 27131, L. reuteri DSM 32465, L. reuteri DSM 33632, L. reuteri DSM 33633, L. reuteri DSM 33634, L. reuteri DSM 33635, L. reuteri DSM 32231, L. reuteri DSM 32232, L. reuteri ATCC PTA 6127 and L. reuteri ATCC PTA 4659.

Alternatively or additionally, the L. reuteri strain as used herein may be provided from at least one L. reuteri strain selected from L. reuteri DSM 17938, L. reuteri ATCC PTA 5289, L. reuteri ATCC PTA 6475, L. reuteri DSM 32846, L. reuteri DSM 32848, L. reuteri DSM 32849, L. reuteri DSM 27131, L. reuteri DSM 32465 or L. reuteri ATCC PTA 4659.

A currently preferred L. reuteri strain is L. reuteri DSM 17938.

Alternatively or additionally, the L. reuteri strain as used herein may be provided from at least one L. reuteri strain selected from L. reuteri ATCC PTA 5289, L. reuteri ATCC PTA 6475, L. reuteri DSM 32846, L. reuteri DSM 32848, L. reuteri DSM 32849, L. reuteri DSM 27131, L. reuteri DSM 32465 or L. reuteri ATCC PTA 4659.

Lactobacillus reuteri DSM 17938 was deposited under the Budapest Treaty at the DSMZ-Deutsche Sammlung von Mikroorganismen und Zellkulturen GmbH (Mascheroder Weg 1b, D-38124 Braunschweig, Germany) on Jan. 30, 2006.

Lactobacillus reuteri DSM 27131 was deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Apr. 18, 2013.

Lactobacillus reuteri DSM 32846, DSM 32847, DSM 32848 and DSM 32849 were deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Jul. 4, 2018.

Lactobacillus reuteri DSM 32465 was deposited under the Budapest Treaty at the Leibniz Institute DSMZ-German collection of Microorganisms and Cell Cultures (Inhoffenstr. 7B, D-38124 Braunschweig, Germany) on Mar. 21, 2017.

Lactobacillus reuteri DSM 33632, DSM 33633, DSM 33634, and DSM 33635 were deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Sep. 9, 2020.

Lactobacillus reuteri DSM 32231 and DSM 32232 were deposited under the Budapest Treaty at the Leibniz Institut DSMZ-Deutsche Sammlung von Mikroorganismen und Zellkulturen GmbH (Inhoffenstr. 7B, D-38124 Braunschweig, Germany) on Dec. 11, 2015.

Lactobacillus reuteri ATCC PTA 5289 was deposited under the Budapest Treaty at the American Type Culture Collection (10801 University Blvd., Manassas, VA 20110-2209, U.S.) on Jun. 25, 2003.

Lactobacillus reuteri ATCC PTA 6475 was deposited under the Budapest Treaty at the American Type Culture Collection (10801 University Blvd., Manassas, VA 20110-2209, U.S.) on Dec. 21, 2004.

Lactobacillus reuteri ATCC PTA 4659 was deposited under the Budapest Treaty at the American Type Culture Collection (10801 University Blvd., Manassas, VA 20110-2209, U.S.) on September 11, 2002.

Lactobacillus reuteri ATCC PTA 6127 was deposited under the Budapest Treaty at the American Type Culture Collection (10801 University Blvd., Manassas, VA 20110-2209, U.S.) on Jul. 22, 2004.

The term “GOS” as used herein means “galacto-oligosaccharide”. Galacto-oligosaccharides (GOS) as used herein typically consist of 6-linked galactose moieties with galactose or glucose at the reducing end. Such GOS contain β-(1→2), β-(1→3), β-(1→4), or β-(1→6) linked galactose moieties and may have a degree of polymerization (DP) of 3-8 galactose units. The term GOS is therefore preferably referred to as oligosaccharide(s) comprising at least three galactose units, more preferably as oligosaccharide(s) comprising at least four galactose units, preferably having a degree of polymerization (DP) of 3-8 galactose units. Oligosaccharides occur naturally in the milk of some mammals, e.g., cow and human. GOS are commercially available and can be synthesized via biosynthesis processes from lactose by trans-galactosidase activity of β-galactosidases. GOS formation in biosynthesis procedures is usually favored by high concentrations of lactose or lactulose, incomplete lactose turnover, low water activity, and the use of enzymes with preference for trans-galactosylation. The linkage type(s) of resulting GOS from such processes is(are) specific for the enzymes used for biosynthesis.

One particular type of mixture of carbohydrates that can advantageously be used herein as a source of GOS to grow the probiotic bacteria is a mixture of GOS and of cow's milk oligosaccharides. In particular mixtures of GOS with 3′-sialyllactose (3′-SL) and/or 6′-sialyllactose (6′-SL) are preferably used. Indeed, such mixtures contain some oligosaccharides similar to human milk, which is particularly advantageous when the composition of the invention is used as an infant formula or as a nutritional supplement for infants. Such advantageous effects are described for example in Simeoni et al.; “Gut microbiota analysis reveals a marked shift to bifidobacteria by a starter infant formula containing a synbiotic of bovine milk-derived oligosaccharides and Bifidobacterium animalis subsp. lactis CNCM I-3446”; Environ Microbiol, 2016, 18(7): 2185-2195. Such compositions can typically be obtained from concentrating whey permeate to obtain a concentrated bovine milk oligosaccharide composition and either adding GOS or generating the GOS in situ from hydrolysis of lactose by the action of a β-galactosidase.

The GOS source used in step ai) of the inventive method to pre-condition the probiotic L. reuteri strain and the GOS source further added in step b) for preparing a final composition comprising the pre-conditioned probiotic L. reuteri strain with GOS, can be provided in the form of essentially pure GOS (i.e., ingredient having at least 90% GOS) or as part of a mixture, such as a mixture of carbohydrates, comprising GOS.

One aspect for the pre-conditioning of a probiotic L. reuteri strain with GOS is that a sufficient amount of GOS is provided in the growth medium. According to one embodiment, the GOS source is therefore typically added to the growth medium in step ai) of the inventive method for preparing a probiotic composition in an amount such as providing at least 0.2 wt %, preferably at least 0.5 wt %, even more preferably at least 0.75 wt % of GOS in the growth medium. In a preferred embodiment, GOS is provided in an amount such as providing at most 8 wt % of GOS, such as about 7 wt % GOS, 6 wt % GOS, or even 5 wt % GOS at most, more preferably at most 4 wt % of GOS or even at most 3 wt % GOS, in the growth medium, preferably calculated on basis of the growth medium (% w/w). Any combination of these upper and lower ranges is encompassed herewith. Preferably, GOS is added to the growth medium in a range of 0.2 wt % to 8 wt % or even 0.2 wt % to 7 wt %, or 0.2 wt % to 6 wt %, such as in a range of 0.5 wt % to 8 wt % or even 0.5 wt % to 7 wt %, or 0.5 wt % to 6 wt %, or in a range of 0.75 wt % to 8 wt % or even 0.75 wt % to 7 wt %, or 0.75 wt % to 6 wt %. Preferably, GOS is added to the growth medium in a range of 0.2 wt % to 8 wt %, preferably in a range of 0.5 wt % to 7 wt %, more preferably in a range of 0.75 wt % to 6 wt %, and most preferably in a range of 0.75 wt % to 5 wt %, such as a range of 0.75 wt % to 3 wt % or 0.75 wt % to 4 wt %, preferably calculated on basis of the growth medium.

When a mixture of carbohydrates is used as a source of GOS for pre-conditioning a probiotic L. reuteri strain or when preparing the final composition containing the pre-conditioned probiotic L. reuteri strain with GOS, it is preferred that the amount of GOS in such a mixture is at least 20 wt %, preferably at least 30 wt %, more preferably at least 40 wt %, even more preferably at least 45 wt %, most preferably at least 48 wt %, preferably calculated on a dry weight basis of the mixture containing GOS.

In an embodiment, GOS is used use as substrate, such as fermentation substrate, for the probiotic L. reuteri strain in step ai). Hence, in such an embodiment, GOS is used as a carbon source during cultivation or fermentation of the probiotic L. reuteri strain.

Although it is not necessary that the bacteria consume only GOS as carbon source during the fermentation, high proportion of GOS in the GOS source during the pre-conditioning step favors the consumption of GOS by the bacteria during fermentation over consumption of other carbohydrates, leading to improved pre-conditioning effect. Smaller carbohydrates such as di-saccharides (lactose for instance) can also be present and be consumed by the bacteria supporting the pre-conditioning effect. Accordingly, it is also preferred that during the pre-conditioning step ai) the GOS source comprises at most 55 wt %, preferably at most 50 wt %, more preferably at most 45 wt %, most preferably at most 42 wt % of mono- or di-saccharides and/or no more than 40 wt %, preferably no more than 30 wt % lactose, preferably calculated on a dry weight basis of the mixture containing GOS. As an example, a mixture of carbohydrate used as a source of GOS during the pre-conditioning step may comprise GOS in amounts defined above for the mixture and the remainder formed by lactose, glucose, and/or galactose, and optionally 3′-SL and/or 6′-SL.

In an embodiment, the culture medium also comprises an electron acceptor, such as fructose, citrate, glycerol and/or 1,2-propanediol.

In a particular embodiment, the culture medium also comprises fructose, such as acting as an electron acceptor. In illustrative, but non-limiting, examples the culture medium may comprise at least 0.2 wt % fructose, preferably at least 0.5 wt % fructose, more preferably at least 1 wt % fructose, even more preferably at least 1.5 wt % fructose calculated on a dry weight basis of the culture medium. Correspondingly, in illustrative, but non-limiting, examples the culture medium preferably does not comprise more than 16 wt % fructose, preferably no more than 14 wt % fructose, more preferably no more than 12 wt % fructose and even more preferably no more than 10 wt % fructose. Any combination of these upper and lower ranges is encompassed herewith. In an embodiment, the culture medium comprises a wt % ratio between the electron acceptor (e.g., fructose) and GOS selected within a range of from 0.25:1 to 5:1, preferably within a range of from 0.5:1 to 4:1, and more preferably within a range of from 0.75 to 2.5:1. Particularly, preferred wt % ratios between the electron acceptor (e.g., fructose) and GOS are within a range of from about 1:1 to about 2:1.

According to a further embodiment, addition of GOS in step ai) of the method for preparing a probiotic composition to the growth medium may occur at any point of time of the cultivation step ai), e.g., at the start of cultivation of probiotic L. reuteri strain, in the middle or at the end of cultivation of probiotic L. reuteri strain in step ai), wherein addition may occur either at once, stepwise, e.g., at multiple separate time instances, or continuously.

The cultivation step ai) is carried out in a way that is well known to the person skilled in the art. Cultivation in step ai) includes the steps of inoculation of sterile standard growth medium with a defined amount of bacteria (colony-forming unit (cfu)), followed by incubation under defined temperature (usually 37° C.) and pH. In this context, a defined amount of bacteria (cfu) for starting an inoculation of sterile standard growth medium may be determined by a skilled person according to common general knowledge and practice in the art. Suitable yields can be obtained with the growth medium comprising GOS as described above, preferably without changing the cultivation conditions compared to what the person skilled in the art would use for cultivation of the same strain with a standard growth medium.

A standard growth medium for cultivating/fermenting the L. reuteri strain in step ai) of the current invention may be any known standard growth medium used for lactobacilli and lactic acid bacteria (LAB), such as MRS medium, or any further suitable medium. Such a standard growth medium is preferably commercially available but may be also produced by addition of compounds according to well-known standard recipes. A standard growth medium for the L. reuteri strain may typically contain carbohydrates, such as simple sugars selected e.g., from dextrose, sucrose, maltose, fructose or lactose, various nitrogen sources, such as peptone, yeast extract, beef extract, or whey protein, minerals, mainly Mn²⁺ and Mg²⁺, and buffering agents, such as sodium acetate (CH₃COONa), trisodium citrate (Na₃C₆H₅O₇), or disodium-glycerophosphate (C₃H₇Na₂O₆P) are commonly used buffers in LAB media. Other components that have been used in standard growth media with buffering activity may include disodium phosphate (Na₂HPO₄), ammonium citrate (NH₄C₆H₅O₇), trisodium phosphate (Na₃PO₄), potassium biphosphate (KH₂PO₄), magnesium phosphate tribasic Mg₃(PO₄)₂, calcium carbonate (CaCO₃), and dipotassium phosphate (K₂HPO₄). Such standard growth media may also contain a wide range of growth factors, surfactants, such as lecithin or Tween® (e.g., Tween® 20, 80 and 85). The cultivation/fermentation may be carried out under anaerobic or aerobic conditions, depending on the strain to be produced, but preferably under anaerobic conditions. Also, the pH may be controlled or not, depending on the conditions known to be the best for a specific strain to grow. The temperature and duration of the cultivation step is variable from one strain to another and is also well-known to the person skilled in the art of probiotic the L. reuteri strain cultivation.

According to one embodiment the cultivation/fermentation of the probiotic L. reuteri strain in step ai) of the inventive method occurs over a considerable period of time to allow a pre-conditioning of the probiotic L. reuteri strain in the presence of GOS, typically for a period of at least 1 hour, preferably for a period of between 5 to 18 hours, likewise preferably 7 to 14 hours, more preferably for a period of between 10 to 13 hours, and even more preferably for a period of about 12 hours.

Moreover, according to a further embodiment the cultivation/fermentation of the probiotic L. reuteri strain in step ai) of the inventive method occurs at a temperature of between 25 to 45° C., preferably between 30 and 40° C., and even more preferably at a temperature of between 35 and 39° C., such as about 37° C.

Additionally, in another embodiment the cultivation/fermentation of the probiotic L. reuteri strain in step ai) of the inventive method occurs at a pH of between 3.0 and 7. The pH during pre-conditioning of probiotic L. reuteri strain during cultivation step ai) may be either not controlled or set to a constant value throughout the cultivation step ai), typically to be monitored and adjusted during cultivation step ai). Accordingly, the pH may be between 3.0 and 7, particularly when pH is not controlled during pre-conditioning of probiotic L. reuteri strain. Alternatively, if pH is controlled during cultivation step ai), pH may be adjusted to a range of about 5.0 to 7.0, e.g., about 5.0 to 6.0, about 5.5 to 6.5 (e.g., about 6.0±0.2) or about 6.0 to 7.0 (e.g., about 6.6±0.2), more preferably pH may be adjusted to a range of about 5.5 to 6.5, even more preferably pH may be adjusted to a range of about 6.0±0.4, and most preferably pH may be adjusted to a range of about 6.0±0.2, likewise preferably pH may be adjusted to a range of about 6.0 to 7.0, likewise more preferably about 6.6±0.4, most preferably 6.6±0.2.

After cultivation/fermentation of a probiotic L. reuteri strain in step ai) of the inventive method the thereby pre-conditioned probiotic L. reuteri strain is harvested in step aii). The harvesting step, which aims at separating the bacterial cells from the growth medium, is also carried out in a way that is well known to the person skilled in the art, such as by concentrating the bacteria, centrifugation, filtration, membrane-filtration, decantation, isolation, purification, etc., preferably centrifuging or concentrating the probiotic L. reuteri strain. Typically, harvesting of the pre-conditioned probiotic L. reuteri strain occurs in step aii) after a period of at least 1 hour as defined above for cultivation/fermentation in step ai).

After harvesting the pre-conditioned probiotic L. reuteri strain in step aii), the harvested cells may be washed in an optional step aiii) prior to further processing, typically to remove traces of growth medium (after cultivation/fermentation). Washing may occur with either saline water, brine, or any further suitable liquid.

Either directly after harvesting the pre-conditioned probiotic L. reuteri strain from the growth medium according to step aii) or after an optional washing step aiii) the pre-conditioned probiotic L. reuteri strain may be subjected to a drying step aiv). Such a drying step aiv) may be carried out by any method known to a skilled person, such as spray-drying, fluid bed drying, air convective drying, atmospheric drying, roller drying or freeze drying, lyophilizing, and more preferably spray-drying or freeze drying. Spray drying may also be carried out in the presence of protecting agents, e.g., as described in WO 2017/001590.

Optionally, the pre-conditioned probiotic L. reuteri strain obtained after harvesting from the growth medium and/or after washing the pre-conditioned probiotic L. reuteri strain may further be mixed with protective agents, such as protectants, cryoprotectants, lyoprotectants and/or carriers before the drying step, as known to the person skilled in the art and as appropriate depending on the drying method to be used and the bacteria to be dried. Such protectants, cryoprotectants, lyoprotectants and/or carriers typically include glycerol, skimmed milk, serum albumin, peptone, yeast extract, saccharose, glucose, sorbitol, malt extract, trehalose etc. Commercially available protectants or cryoprotectants include e.g., Unipectine™ RS 150, etc. or are as described in EP 3016511, US 2014/0004083 A1, US 2016/0298077 A1, etc.

Alternatively, or additionally, the pre-conditioned probiotic L. reuteri strain obtained after harvesting from the growth medium, after washing or even after drying, i.e. obtained after any of steps aii) or aiii) after aiv), may be encapsulated for further preservation and/or use, e.g., by formation of microspheres containing the pre-conditioned probiotic L. reuteri strain using common encapsulation agents, e.g., alginate, alginate/pullulan, starch, xanthan, xanthan-gellan gum mixtures, gelatin, cellulose acetate phthalate, chitosan, or any further suitable encapsulation material. Encapsulation is well known and can be carried out by a person skilled in the art.

Furthermore, the inventive method for preparing a probiotic comprises the step b) of adding GOS to the pre-conditioned probiotic L. reuteri strain obtained from step a) to prepare, produce or form the probiotic composition. GOS may be applied in amounts as defined above, either pure or as a mixture as defined before.

The prebiotic GOS may be added to the pre-conditioned probiotic L. reuteri strain any time following culturing in step ai). If the method comprises additional steps as mentioned above, i.e., washing and/or drying, GOS could be added prior to, during and/or following any such steps. In a particular embodiment, GOS is added to the pre-conditioned probiotic L. reuteri strain following the drying step.

GOS could be added to the pre-conditioned probiotic L. reuteri strain once or at multiple different occasions during the method.

According to a preferred embodiment, GOS may be added in step b) of the inventive method to the pre-conditioned probiotic L. reuteri strain obtained from step a) in an amount of at least 1 or 2 g, preferably at least 3 g per, likewise preferably between 2 to 12 g per, or between 2 to 10 g per, between 2 to 8 g per, or between 2 to 7 g, more preferably between 3 to 12 g, likewise more preferably between 3 and 10 g, such as between 3 to 8 g, between 3 to 7 g, between 3 to 6 g, or about 2, 3, 4, 5, 6, 7, 8, 9, 10, 11, or 12 g or any range formed thereby, to prepare the (final) probiotic composition. The above mentioned amounts of GOS added in step b) are preferably amount of GOS per daily dose. Hence, in a preferred embodiment, the probiotic composition is provided in a dosage form or unit, which is further described herein. In such a case, such a dosage form or unit preferably comprises the above mentioned amount of GOS. For instance, a dosage unit of the probiotic composition comprises at least 1 g GOS. This means that step b) then corresponds to adding GOS to the pre-conditioned probiotic L. reuteri strain obtained from step aii) to prepare a dosage unit of the probiotic composition and where this dosage unit comprises at least 1 g GOS.

### Compositions Comprising the Pre-Conditioned Probiotic L. reuteri Bacterium and GOS

According to the second aspect, the invention provides a probiotic composition comprising an effective amount of a pre-conditioned probiotic L. reuteri strain and additionally an effective amount of GOS, wherein the pre-conditioned probiotic L. reuteri strain has been prepared by cultivating a L. reuteri strain in the presence of GOS in a growth medium.

In an embodiment, the probiotic composition has been prepared/obtained according to the inventive method, i.e., is obtainable by the inventive method for preparing a probiotic composition. In this context, a pre-conditioned probiotic L. reuteri strain is a probiotic L. reuteri strain that has been cultivated in the presence of GOS, e.g., under conditions as defined above for the inventive method for preparing a probiotic composition. Such a pre-conditioning of a probiotic L. reuteri strain in the presence of GOS is typically carried out according to the inventive method as described herein under the first aspect of the invention. As already disclosed above in detail, cultivating/fermentation of a probiotic L. reuteri strain in the presence of GOS preferably occurs over a time sufficient to pre-condition a probiotic L. reuteri strain accordingly, e.g., over a period of at least one hour, preferably according to a process as defined herein.

The probiotic composition comprises an effective amount of a harvested, pre-conditioned L. reuteri strain and an effective amount of GOS added or supplemented, i.e., in the form of a GOS supplement, to the harvested, pre-conditioned L. reuteri strain.
As before, the L. reuteri strain is particularly preferably at least one L. reuteri strain or multiple L. reuteri strains as defined above, preferably selected from the group comprising or consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 27131, L. reuteri DSM 32465, L. reuteri DSM 33632, L. reuteri DSM 33633, L. reuteri DSM 33634, L. reuteri DSM 33635, L. reuteri DSM 32231, L. reuteri DSM 32232, L. reuteri ATCC PTA 6127 and L. reuteri ATCC PTA 4659, more preferably selected from the group comprising or consisting of L. reuteri DSM 17938, L. reuteri ATCC PTA 5289, L. reuteri ATCC PTA 6475, L. reuteri DSM 32846, L. reuteri DSM 32848, L. reuteri DSM 32849, L. reuteri DSM 27131, L. reuteri DSM 32465 and L. reuteri ATCC PTA 4659, and most preferably the probiotic L. reuteri strain is L. reuteri DSM 17938.

In one embodiment, the L. reuteri strain is particularly preferably at least one L. reuteri strain or multiple L. reuteri strains as defined above, preferably selected from group comprising or consisting of L. reuteri DSM 17938, L. reuteri ATCC PTA 5289, L. reuteri ATCC PTA 6475, and L. reuteri ATCC PTA 4659.

In one embodiment, the L. reuteri strain is particularly preferably at least one L. reuteri strain or multiple L. reuteri strains as defined above, preferably selected from group comprising or consisting of L. reuteri DSM 32846, L. reuteri DSM 32848, L. reuteri DSM 32849, L. reuteri DSM 27131, and L. reuteri DSM 32465.

Typically, an “effective amount” of a pre-conditioned probiotic L. reuteri strain as defined herein for the composition may comprise a pre-conditioned probiotic L. reuteri strain typically in an amount of between 10³ cfu to 10 12 cfu, typically in an amount of between 10⁴ cfu to 10¹¹ cfu, preferably in an amount of between 10⁵ cfu to 10¹⁰ cfu or 10⁵ cfu to 10⁹ cfu, likewise preferably in an amount of between 10⁶ cfu to 10⁹ cfu, 10⁶ cfu to 10⁸ cfu or in an amount of 10⁸ cfu to 10¹⁰ cfu. The above mentioned amounts of pre-conditioned probiotic L. reuteri strain are preferably amount of pre-conditioned probiotic L. reuteri strain per daily dose. For instance, a dosage form or unit of the probiotic composition preferably comprises the above mentioned amount of pre-conditioned probiotic L. reuteri strain.

A preferred daily dose is around 10⁸ total cfu per daily dose, e.g., 10⁷ to 10⁹ or 10⁸ to 10⁹ cfu per daily dose.

Moreover, an “effective amount” of GOS as defined herein for the composition may comprise GOS in an amount of at least 1 or 2 g, preferably at least 3 g, likewise preferably between 2 to 12 g, or between 2 to 10 g, between 2 to 8 g, or between 2 to 7 g, such as between 3 to 12 g, likewise more preferably between 3 and 10 g, such as between 3 to 8 g, between 3 to 7 g, between 3 to 6 g, or about 2, 3, 4, 5, 6, 7, 8, 9, 10, 11, or 12 g or any range formed thereby. The above mentioned amounts of GOS are preferably amount of GOS per daily dose. For instance, a dosage form or unit of the probiotic composition preferably comprises the above mentioned amount of GOS.

The probiotic composition can comprise the pre-conditioned probiotic L. reuteri strain in dried form as previously mentioned herein. In such a case, the pre-conditioned probiotic L. reuteri strain is preferably in spray-dried or freeze dried form. Hence, in an embodiment, the probiotic composition comprises an effective amount of a freeze dried, pre-conditioned probiotic L. reuteri strain and the effective amount of GOS.

The inventive probiotic composition comprising an effective amount of a pre-conditioned probiotic L. reuteri strain and additionally an effective amount of GOS, as defined herein may be any type of composition, in which probiotic bacteria can be incorporated, such as a food product, a beverage, an animal feed product, a nutritional supplement for human or animal, a pharmaceutical composition or a cosmetic composition. More preferably, the inventive probiotic composition may be provided in an administrable form, preferably selected from the group consisting of food products, beverages, pharmaceutical formulations, dietary supplements, nutritional compositions or supplements, functional food products, functional beverage products, nutraceuticals, and combinations thereof. Illustrative examples of such administrable form include dairy products, milk-based products, and whey protein-based beverages.

According to one particular embodiment, the effective amount of a pre-conditioned probiotic L. reuteri strain and the effective amount of GOS may be contained in two different parts of a kit-of-parts. The method or preparation as described before is then carried out as described before with the mere difference that the effective amount of GOS is added to a different part of the kit-of-parts than the effective amount of a pre-conditioned probiotic L. reuteri strain.

The inventive probiotic composition may be solid or liquid. Preferably it is present in form of a powder, a sachet, a tablet, a capsule, or may be in form of an oil formulation, an emulsion, e.g., an oil-in-water emulsion (o/w emulsion), a water-in oil emulsion (w/o emulsion), etc. If present in powder form it can be intended to be used by the final consumer in solid (such as powder form) or semi-solid form (such as for example in the form of a paste) or, alternatively, to be reconstituted into a liquid before use.

Food products and beverages as defined herein may include all products intended to be consumed orally by human beings, for the purpose of providing nutrition and/or pleasure. The expression “food product” as well as the term “beverages” usually mean compositions, which nourish a subject. This “food product” is usually to be taken orally or intraperitoneally, and it can include a lipid or fat source and a protein source. Likewise, a “beverage” is usually to be taken orally, is liquid or a semi-liquid, and can include a lipid or fat source and a protein source.

Food products and beverages as defined herein can for example include a nutritional composition, preferably for human consumption, such as for infants and/or young children, for a pregnant or lactating woman or a woman desiring to get pregnant, for individuals in need of a special nutrition due to an adverse health condition or for elderly people. More preferably, the nutritional composition is selected from infant formula, infant cereals, follow-up or follow-on formula, growing-up milks, functional milks, baby food, infant cereal compositions, and milk products for pregnant and lactating women or for women desiring to get pregnant. Other examples of food products and beverages include sweet and savory snacks, powdered drinks, cereal products and dairy products, such as milk products, whey protein-based products, etc. According to one particular embodiment, the inventive probiotic composition is an infant formula, a follow-on formula, a growing-up milk or a product for pregnant or lactating women. In one further particular embodiment, the inventive probiotic composition may be an infant formula.

The inventive probiotic composition can also be in the form of an animal food product or a nutritional supplement for animals. Preferably, the animal is a mammal. Examples of animals include cows, sheep, goats, horses, dogs, cats, rabbits, rats, mice, fish, birds and the like. The preferred use of the inventive probiotic composition is, however, for humans.

The expression “infant formula” as used herein refers to a foodstuff intended for particular nutritional use by infants during the first months of life and satisfying by itself the nutritional requirements of this category of person (reference : Article 2(c) of the European Commission Directive 91/321/EEC 2006/141/EC of 22 Dec. 2006 on infant formulae and follow-on formulae; Reg 609/2013 art 2.1.c and Reg 2016/127). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”, and includes compositions to be provided to premature infants.

A “follow-up formula” or “follow-on formula” is an infant nutritional composition given from the 6th month onwards. It constitutes the principal liquid element in the progressively diversified diet of this category of person.

The expression “baby food” means a foodstuff intended for particular nutritional use by infants or young children during the first years of life.

In the inventive context, the term “infant” means a child under the age of 12 months. Moreover, the expression “young child” means a child aged between one and three years, also called toddler.

The expression “infant cereal composition” means a foodstuff intended for particular nutritional use by infants or young children during the first years of life.

Dietary or nutritional supplements are typically present in the form of a liquid, such as a refrigerated liquid, in form of a powder or a tablet or capsule, an oil formulation, an emulsion, e.g., an oil-in-water emulsion (o/w emulsion), a water-in oil emulsion (w/o emulsion), etc. as mentioned above. Preferably it is in the form of a powder, a sachet, a tablet, a capsule or an oil formulation. Powder supplements typically encompass supplements to be dissolved in a liquid or to be sprinkled on food or in a beverage. Such supplements are intended to provide additional nutrients and/or a health benefit to the subject consuming it, as well as other beneficial ingredients, such as the herein-defined and prepared pre-conditioned probiotic bacteria L. reuteri and additionally an effective amount of GOS. A supplement according to the present invention can therefore be used for providing nutrients and/or a health benefit to human beings, as well as to animals, as defined above. Dietary or nutritional supplements include for example the herein-defined and prepared pre-conditioned probiotic bacteria L. reuteri and additionally an effective amount of GOS as a powder supplement to be added to any sort of dietary or nutritional composition.

Pharmaceutical products include powder, sachet, tablet or capsule products intended to treat or prevent an adverse medical condition in a subject in need thereof, or to promote a favorable health condition.

Cosmetic compositions are typically intended for an aesthetic effect on the body and may be for topical use or may be administered by oral route, in the form of a powder, sachet, tablet or capsule.

### Applications of Inventive Compositions Comprising Pre-Conditioned Probiotic L. reuteri Bacterium and GOS

The present invention aims to provide solutions, compositions and uses, which, among others, increase mineral solubility and bio-accessibility and also mineral absorption in the gastrointestinal tract. In detail, such solutions are probiotic compositions comprising an effective amount of GOS pre-conditioned probiotic L. reuteri strain and additionally an effective amount of GOS to be administered to a subject or a patient in need thereof, e.g., a subject or a patient in need of increased mineral uptake. The increased mineral solubility, accessibility, and absorption takes place in the gastrointestinal tract, such as in the duodenum (first part of small intestine), the jejunum (middle part of small intestine), the ileum (final part of the small intestine) and/or in the colon (large intestine). The present invention also aims to provide methods for preparing probiotic compositions comprising a pre-conditioned probiotic L. reuteri strain and additionally, i.e., supplemented with, an effective amount of GOS such that increased mineral solubility and bio-accessibility and also increased mineral absorption in the gastrointestinal tract can be achieved by virtue of such a combination.

As already outlined above, minerals are essential for all living species, even though the specific requirements differ between species. Mineral deficiencies can lead to diseases and so can mineral excess. Correct amounts of minerals at the correct ratios are, thus, required for optimal health. Most natural diets will provide minerals in appropriate balances, but there are situations when a balanced diet is not enough to maintain health with respect to minerals. As mentioned, minerals are essential and have a great number of important functions in an organism.

Iron, for example, is responsible for the transport of oxygen and myoglobin. Moreover, iron is part of many enzymes, such as catalases, peroxidases and cytochromes. Iron is generally absorbed in the duodenum and the upper part of the small intestine in the form of Fe²⁺ by cells that line the gastrointestinal tract, which absorbs iron from the ingested food.

Calcium is another essential mineral, which is inter alia an important bone component and an enzyme activator. Calcium also takes part in conduction of bioelectric impulses, blood coagulation, muscle contraction, inflammation and hormonal secretion. Calcium is generally absorbed in the small intestine in the presence of the active form of vitamin D (calcitriol).

A mineral deficiency of any of such minerals can lead to severe complications, conditions and diseases. The following uses, both non-therapeutic and therapeutic, are intended to address such mineral deficiency either by preventing or treating mineral deficiencies, e.g., in healthy subjects in a non-therapeutic manner, e.g., by providing the inventive compositions as nutritional supplements or beverage, or in a subject in need thereof by administering or supplying the composition as defined above, e.g., as nutritional composition or beverage, as a pharmaceutical composition, etc.

The present invention also provides methods, solutions, compositions and uses, which increase mineral solubility and bio-accessibility and also mineral absorption in the gastrointestinal tract using a probiotic composition comprising GOS pre-conditioned probiotic L. reuteri strain in combination with an additional amount of GOS. Any of the herein described applications may be carried out with the inventive composition as described above or a kit-of-parts as described herein.

As shown herein inventive probiotic compositions allow for enhancing probiotic effects of L. reuteri strains in a mammal, using GOS as specific substrate components during fermentation when manufacturing the probiotic L. reuteri strains and supplementing such pre-conditioned probiotic L. reuteri strain further with GOS to prepare the final composition. The herein disclosed method specifically provides for the preparation of such a probiotic composition, which pre-conditions in a first step the probiotic L. reuteri strain by use of GOS during cultivation of the probiotic L. reuteri strain, such that after addition of GOS in a second step a particularly increased beneficial probiotic effect of the L. reuteri strain occurs in vivo, particularly during non-therapeutic and therapeutic administrations as disclosed herein. This particularly concerns and allows e.g., an increase in survival of the probiotic L. reuteri strain, an increased metabolic activity of the gut microbiota, and an increase of bio-accessibility of minerals, e.g., by increasing mineral solubility, accessibility and/or absorption of minerals within the gastrointestinal tract of a subject or patient upon administering the composition comprising the pre-conditioned probiotic L. reuteri strain together with GOS, as described herein.

Additionally, an increased metabolic activity of the gut microbiota becomes evident e.g., through an increased lactic acid production upon administering the pre-conditioned probiotic L. reuteri strain supplemented with GOS, as described herein. The observed increased lactic acid together with an increased acetic acid production indicate a higher activity of certain gut bacteria (e.g., Lactobacillus and lactic acid producing bacteria) and shows that the microbiota has been modulated (resulting in a healthier microbiota) due to administration of the pre-conditioned probiotic L. reuteri strain supplemented with GOS. The improved survival of L. reuteri, the significantly increased metabolic activity of colonic microbiota and the particular increase of mineral (iron and calcium) solubility in the colon can be attributed to the combined administration of the pre-conditioned L. reuteri further supplemented with GOS in the final composition. This combination conferred to a subject and/or patient in need thereof by administering the inventive probiotic composition makes it possible to decrease the dosage and/or the frequency of administration of the probiotic required to obtain the sought after health effects.

In the following, without being limiting thereto, exemplary therapeutic and non-therapeutic applications (uses) are listed.

Non-Therapeutic Uses of Compositions Comprising Pre-Conditioned Probiotic L. reuteri Bacterium and GOS

According to the third aspect, the invention provides a non-therapeutic use of a probiotic composition comprising an effective amount of a pre-conditioned probiotic L. reuteri strain and an effective amount of GOS obtainable by or prepared by the inventive method or otherwise as described herein, typically in form of an administrable composition, to improve, increase or boost the probiotic effect of the L. reuteri strain in the digestive tract of a healthy subject. Such an improvement, increase or boosting of the probiotic effect by a pre-conditioned probiotic L. reuteri strain supplemented with GOS in the digestive tract of a healthy subject can be typically to be determined in comparison with the not yet pre-conditioned probiotic L. reuteri strain in the digestive tract of a healthy subject with or without supplementation with GOS.

The improvement, increase or boosting of the probiotic effect, typically of the probiotic L. reuteri strain in the digestive tract of a healthy subject, inter alia but not exclusively concerns the increase of the bio-accessibility of minerals, and specifically the increase of mineral solubility, bio-accessibility and/or absorption of minerals within the gastrointestinal tract of a healthy subject. The increase of the bioavailability of minerals can in one embodiment result in an increased enzymatic function. Such minerals are preferably essential minerals as mentioned herein, e.g., calcium and/or iron.

Increase of the probiotic effect may be manifested e.g., in improving the L. reuteri survival in the intestinal tract of a healthy subject, increasing the lactic acid and/or acetic acid production in the intestinal tract of a subject, and/or promoting a healthy microbiota in a healthy subject.

According to a particular embodiment, a non-therapeutic use of a probiotic composition as defined herein is therefore preferably for improving or increasing the probiotic effect, preferably of a L. reuteri strain, in the digestive tract of a healthy subject. Such an improving or increasing the probiotic effect may be e.g.,
an increase of mineral bio-accessibility, preferably an increase of mineral solubility and/or mineral absorption, in the intestinal tract of the healthy subject; and/or
a promotion of a healthy microbiota in the intestinal tract of the healthy subject; and/or

a promotion of the survival of the L. reuteri strain in the intestinal tract (e.g., in the colon) and, thus, a promotion of the health benefits associated to the L. reuteri strain; and/or
an increase of metabolic activity of the gut microbiota, preferably of the L. reuteri strain, such as in the form of an increase in lactic acid and/or acetic acid production in the intestinal tract of the healthy subject; and/or
is an increase of bone formation and/or bone mineralization and/or an increase of maturation and/or activity of osteoblasts in a healthy subject.

Of the essential minerals, calcium is involved in conduction of bioelectric impulses, blood coagulation, muscle contraction, prevention of inflammation and hormonal secretion. Iron is an important factor in many body functions and processes and is necessary to maintain healthy cells, skin, hair, and nails.

The increase of bio-accessibility of such minerals in the intestinal tract of the healthy subject, preferably by increasing mineral solubility, accessibility and/or absorption in the gastrointestinal tract of the healthy subject thereby preferably allows increasing any of such body functions and processes. These aspects particularly concern non-therapeutical and also cosmetical applications, for which the inventive probiotic compositions may be applied. It is emphasized that healthy subjects can clearly benefit from an improved availability of minerals by the claimed non-therapeutic uses as most minerals are not fully adsorbed by the body. In particular, the healthy subjects can benefit from optimized or improved bone health, bone formation, teeth conditions, teeth growth, muscular functions, cognitive functions, immune functions and/or general growth and development (especially for children/infants).

In addition to such effects on mineral solubility, accessibility and/or absorption of minerals within the gastrointestinal tract of a subject further non-therapeutic uses may encompass an improvement of the L. reuteri strain metabolic activity and/or L. reuteri strain survival in the intestinal tract of a healthy subject, and/or promotion of a healthy microbiota in a healthy subject.

A healthy microbiota in a subject as used herein means that the composition of the microbiota in the subject is favorable and beneficial for the subject. As a consequence, the probiotic composition of the invention can result in a favorable microbiota composition (“healthy microbiota”) in the subject and may even lead to a more favorable microbiota composition (“healthier or more healthy microbiota”) in the subject.

In this context, a healthy subject, which is the main addressee of this non-therapeutic use, may be selected from an athlete, a child, a toddler or an infant, an adult, an elderly, a pregnant woman, a vegetarian, a lactating woman, a companion animal, a cat, or a dog, or a bird, preferably for addressing or even improving such body functions and body processes. Such a healthy subject usually does not suffer from any condition or disease, particularly not from any mineral deficiency as described herein.

Any of the embodiments as discussed herein for pre-conditioning a probiotic L. reuteri strain as well as any of the features of probiotic compositions as defined herein comprising the pre-conditioned probiotic L. reuteri strain supplemented with GOS apply mutatis mutandis for the third aspect of the current invention, namely to the non-therapeutic use of the inventive probiotic compositions as described above.

This specifically concerns features of the method of pre-conditioning a probiotic L. reuteri strain, the preparation of the probiotic composition with the pre-conditioned probiotic L. reuteri strain supplemented with GOS and any ingredients and amounts and further features as used for compositions comprising the pre-conditioned probiotic L. reuteri strain. The probiotic composition may be provided in any form as depicted above.

In this context, it is e.g., noted that the inventive probiotic composition for non-therapeutic purposes may be solid or liquid, and/or is present in form of a powder, a sachet, a tablet, a capsule, or may be in form of an oil formulation, an emulsion, an oil-in-water emulsion (o/w emulsion), or a water-in oil emulsion (w/o emulsion).

Moreover, the inventive probiotic composition for non-therapeutic purposes may be in an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage.

As before, the L. reuteri strain is particularly preferable at least one L. reuteri strain or multiple L. reuteri strains selected from the group comprising or consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 27131, L. reuteri DSM 32465, L. reuteri DSM 33632, L. reuteri DSM 33633, L. reuteri DSM 33634, L. reuteri DSM 33635, L. reuteri DSM 32231, L. reuteri DSM 32232, L. reuteri ATCC PTA 6127 and L. reuteri ATCC PTA 4659, preferably selected from the group comprising or consisting of L. reuteri DSM 17938, L. reuteri ATCC PTA 5289, L. reuteri ATCC PTA 6475, L. reuteri DSM 32846, L. reuteri DSM 32848, L. reuteri DSM 32849, L. reuteri DSM 27131, L. reuteri DSM 32465 and L. reuteri ATCC PTA 4659, and more preferably the probiotic L. reuteri strain is L. reuteri DSM 17938.

### Use of Compositions Comprising Pre-Conditioned Probiotic L. reuteri Bacterium and GOS as a Medicament

According to the fourth aspect, the invention also provides a probiotic composition comprising an effective amount of a pre-conditioned probiotic L. reuteri strain supplemented with GOS obtainable by the inventive method or otherwise as described herein for use as a medicament.

Similar as before, the effect of such a probiotic composition as a medicament is particularly related to the increase of the synbiotic pre- and probiotic effects in the digestive tract of a subject in need of such a medicament, usually compared to the effect of a probiotic L. reuteri strain not pre-conditioned with GOS or usual combinations of pre- and probiotics.

The therapeutic effects that are related to the use of the inventive probiotic compositions as a medicament inter alia address effects related to any of the herein mentioned diseases and applications, particularly in subjects that are at risk or that are already suffering from any of such diseases, particularly diseases as mentioned explicitly herein in the context of therapeutic applications (see also below).

Any of the embodiments as discussed before for pre-conditioning a probiotic L. reuteri strain as well as any of the features of probiotic compositions as defined herein comprising the pre-conditioned probiotic L. reuteri strain supplemented with GOS also apply mutatis mutandis for the fourth aspect of the current invention, namely to the use of the inventive probiotic compositions as a medicament.

This specifically concerns features of the method of preparing a probiotic composition comprising a pre-conditioned probiotic L. reuteri strain supplemented with GOS, and any ingredients and amounts and further features as used for the inventive probiotic compositions in any form as depicted above.

In this context, it is particularly preferred that the inventive probiotic composition for use as a medicament may be solid or liquid, may be present in form of a powder, a sachet, a tablet, a capsule, or may be in form of an oil formulation, an emulsion, an oil-in-water emulsion (o/w emulsion), or a water-in oil emulsion (w/o emulsion).

Likewise, the inventive probiotic composition for use as a medicament may be in an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage.

As before, the L. reuteri strain is particularly preferable at least one L. reuteri strain or multiple L. reuteri strains selected from the group comprising or consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 27131, L. reuteri DSM 32465, L. reuteri DSM 33632, L. reuteri DSM 33633, L. reuteri DSM 33634, L. reuteri DSM 33635, L. reuteri DSM 32231, L. reuteri DSM 32232, L. reuteri ATCC PTA 6127 and L. reuteri ATCC PTA 4659, preferably selected from the group comprising or consisting of L. reuteri DSM 17938, L. reuteri ATCC PTA 5289, L. reuteri ATCC PTA 6475, L. reuteri DSM 32846, L. reuteri DSM 32848, L. reuteri DSM 32849, L. reuteri DSM 27131, L. reuteri DSM 32465 and L. reuteri ATCC PTA 4659, and more preferably the probiotic L. reuteri strain is L. reuteri DSM 17938.

### Therapeutic Uses of Compositions Comprising Pre-Conditioned Probiotic L. reuteri Bacterium and GOS

According to a fifth aspect, the invention provides a probiotic composition comprising an effective amount of a pre-conditioned probiotic L. reuteri strain supplemented with GOS obtainable by the inventive method or otherwise as described herein for use in the prevention and/or treatment of a disease in a subject in need thereof or for reducing or inhibiting the risk of such a disease.

As used herein, the terms “ treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of subjects at risk of contracting a disease or suspected to have contracted a disease, as well as subjects who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, etc. The terms “treatment” and “treat” further include reducing the risk of developing a disease, e.g., in the form of preventive treatment. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

According to one embodiment, a disease in a subject in need thereof may concern mineral deficiency. Accordingly, a subject in need thereof is also typically a subject that is at risk of developing mineral deficiency or is a subject already suffering from mineral deficiency. Such a subject may be any subject group as described above herein.

In an embodiment, such a subject in need thereof may also be a subject at risk of suffering from a digestive dysfunction, an impaired microbiota, and/or has a digestive dysfunction, an impaired microbiota or a condition requiring promotion of the development and/or growth of the microbiota.

Mineral deficiency in this particular context of the invention preferably concerns a deficiency in calcium and/or iron. Thereby, it is not necessarily a deficiency of just one mineral alone that may lead to a specific disease as depicted below but more often a combined lack of different minerals, e.g., one or more minerals selected from calcium and/or iron, and, consequently, also an overlap in the physiological consequences and a combination of different diseases/conditions that may occur.

As mentioned before, iron deficiency is a condition, which occurs when the body doesn't have enough of the mineral iron. Iron deficiency often leads to abnormally low levels of red blood cells and a condition referred to as iron deficiency anemia. As a result, iron deficiency anemia results in tiredness and shortness of breath. Iron is also known to be very important in maintaining many body functions and is necessary to maintain healthy cells, skin, hair, and nails.

The following groups of people are at highest risk for iron-deficiency anemia and are, hence, suitable for receiving the GOS supplemented pre-conditioned L. reuteri strain composition; women who menstruate, particularly if menstrual periods are heavy, women who are pregnant or breastfeeding or those who have recently given birth, people who have undergone major surgery or physical trauma and lost blood, people with gastrointestinal diseases such as celiac disease, inflammatory bowel diseases (IBD), such as ulcerative colitis (UC), or Crohn's disease, people with peptic ulcer disease, people who have undergone bariatric procedures, especially gastric bypass operations, vegetarians, vegans, and other people whose diets do not include iron-rich foods, children who drink more than 16 to 24 ounces a day of cow's milk (Cow's milk not only contains little iron, but it can also decrease absorption of iron and irritate the intestinal lining causing chronic blood loss.), and infants especially those who have low birth weight or are born prematurely, and/or those who don't get enough iron from breast milk or formula. In general, children need extra iron during growth spurts. Any of such patient groups may be treated herein.

Thus, in one embodiment the inventive probiotic composition obtainable by the inventive method or otherwise as described herein used to treat iron deficiency, preferably iron deficiency anemia, in a subject.

Further, chronic iron deficiency is a known emerging risk factor for bone loss and osteoporosis.

Calcium is another essential mineral, which is important bone component and an activator of enzymes. Calcium also takes part in conduction of bioelectric impulses, blood coagulation, muscle contraction, inflammation and hormonal secretion.

In one embodiment, the present invention therefore provides a probiotic composition obtainable by the inventive method or otherwise as described herein for use in the prevention and/or treatment of calcium deficiency in a subject, for use in the prevention and/or the treatment of bone loss, or for use in the treatment of osteoporosis and/or osteopenia.

Moreover, and particularly in view of calcium deficiencies, further conditions and/or diseases may be prevented or treated related to muscle problems, e.g., cramps, muscle spasms, and aches, fatigue, including insomnia, sleepiness, and extreme fatigue, skin and nail symptoms, eczema, psoriasis, redness, itchiness, skin blisters, osteoporosis and osteopenia, painful premenstrual syndrome, dental problems, depression, etc. Moreover, calcium deficiency (hypocalcemia) is also serious problem in early infancy, which makes infants of said stage to a particular target group. This includes neonatal hypocalcemia, which occurs in the first 2 to 3 days of a baby's life, as well as late hypocalcemia, which starts in the first week or weeks after birth. Any of such diseases may be treated. According to one embodiment, the present invention particularly provides a composition comprising a pre-conditioned L. reuteri strain for use in the promotion of cognitive functions, promotion of oxygen transport, promotion of immune functions, and/or reduction of tiredness or fatigue, in a subject in need thereof.

Populations at risk for calcium deficiency and preferred subject to such a prevention and/or treatment particularly include pregnant women (especially in the last trimester), lactating women, postmenopausal women, older adults, teenagers, and/or people who are overweight. Populations at risk for hypomagnesemia particularly include diabetes patients, patients at risk of diabetes, obese subjects and/or overweight subjects, etc. Any of such subjects and patient groups may be treated herein.

An embodiment relates to a probiotic composition comprising an effective amount of a pre-conditioned probiotic L. reuteri strain and an effective amount of GOS obtainable by the inventive method or otherwise as described herein for use in the prevention and/or treatment of an impaired microbiota or a dysbiosis.

The probiotic composition of the invention leads to a healthier microbiota as observed by an increase in lactic acid and/or acetic acid production. Such an increase in the production of lactic and/or acetic acid indicates a healthier and more metabolic active gut microbiota, in particular Lactobacillus and lactic acid producing bacteria. This means that the probiotic composition of the invention can be used to prevent and/or treat an impaired composition of the microbiota or a dysbiosis in a subject.

A further positive effect of the herein used pre-conditioned L. reuteri strain supplemented with GOS is an improved composition of the microbiota and also an increase in the lactic acid and/or acetic acid production in the microbiota. The increased production of lactic acid and/or acetic acid is effective in prevention and/or treatment of infections, in particular gut infections. Lactic acid and acetic acid are strong anti-pathogenic molecules. Increasing their levels in the colon may increase the protection against pathogens and infections, at least partly by lowering the pH. Hence the probiotic composition according to the invention, in one embodiment, may promote defense against infections, especially gut infections and promote general immune function.

In addition, the increase in survival and/or engraftment and/or viability of a L. reuteri strain in the intestinal tract of a subject to be treated may specifically and positively influence the treatment of any of the herein-mentioned diseases.

The medical uses herein also concern the prevention and/or the treatment of bone loss, the promotion of promote growth, the development of bones or teeth in children, teeth growth, promotion of muscular functions, promotion of functions of the nervous system and/or bone or muscular related conditions, in a subject in need thereof.

The prevention and/or the treatment of bone loss and/or bone loss related disease in a subject in need thereof, is, at least partly, manifested through promoting the bone formation and/or bone mineralization and/or bone strength. The probiotic composition also increased calcium bio-accessibility, such as by increasing calcium solubility and/or calcium absorption. These effects of the probiotic composition is of additional benefit in treatment of bone loss and/or bone loss related diseases.

Hence, in an embodiment, the probiotic composition comprising an effective amount of a pre-conditioned probiotic L. reuteri strain and an effective amount of GOS obtainable by the inventive method or otherwise as described herein can be used in promoting growth and/or development of bone or teeth in children.

Additionally or alternatively, the composition of the invention can be used in the prevention and/or treatment of a digestive dysfunction. Such conditions may include e.g., any treatment or disease that diminishes the amount and/or function of (beneficial) bacteria in the microbiota, such as immunotherapies, cancer therapies, administration of antibiotics, subjects suffering from diarrhea, from infections or inflammations, but may also and particularly concern any such and further conditions related to an impaired microbiota or a condition requiring promotion of the development and/or growth of the microbiota that affect infants and small children.

Generally, subject for any such treatments or a subject in need of such a treatment is a mammal or a bird, preferably, it is human, more preferably it is selected form a child, a toddler or an infant, an elderly, a pregnant woman, a vegetarian, a lactating woman, a companion animal, but also companion pets such as a cat or a dog.

Also, in this context, any of the embodiments as discussed before for preparing a probiotic composition comprising a pre-conditioned probiotic L. reuteri strain supplemented with GOS as well as any of the features of compositions as defined herein comprising the pre-conditioned probiotic L. reuteri strain also apply mutatis mutandis for the fifth aspect of the current invention, namely to the medical or therapeutic uses of the inventive probiotic compositions in any of the herein defined treatments.

This specifically concerns features of the method of preparing a probiotic composition comprising a pre-conditioned probiotic L. reuteri strain supplemented with GOS, and any ingredients and amounts and further features as used for compositions comprising the pre-conditioned probiotic L. reuteri strain. The composition may be provided in any form as depicted above.

In this context, it is particularly preferred that the inventive probiotic composition for medical or therapeutic uses as described herein may be solid or liquid, may be present in form of a powder, a sachet, a tablet, a capsule, or may be in form of an oil formulation, an emulsion, an oil-in-water emulsion (o/w emulsion), or a water-in oil emulsion (w/o emulsion).

Likewise, the inventive probiotic composition for medical or therapeutic uses as described herein may be in an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage.

As before, the L. reuteri strain is particularly preferable at least one L. reuteri strain or multiple L. reuteri strains selected from the group comprising or consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 27131, L. reuteri DSM 32465, L. reuteri DSM 33632, L. reuteri DSM 33633, L. reuteri DSM 33634, L. reuteri DSM 33635, L. reuteri DSM 32231, L. reuteri DSM 32232, L. reuteri ATCC PTA 6127 and L. reuteri ATCC PTA 4659, preferably selected from the group comprising or consisting of L. reuteri DSM 17938, L. reuteri ATCC PTA 5289, L. reuteri ATCC PTA 6475, L. reuteri DSM 32846, L. reuteri DSM 32848, L. reuteri DSM 32849, L. reuteri DSM 27131, L. reuteri DSM 32465 and L. reuteri ATCC PTA 4659, and more preferably the probiotic L. reuteri strain is L. reuteri DSM 17938.

According to a further particular embodiment, the current invention also provides a method of treatment of a disease in a subject in need thereof. Such a disease and a subject are preferably as described herein. The method of treatment preferably includes the step of administering to the subject a probiotic composition of the invention, i.e., administering to the subject an effective amount of a pre-conditioned probiotic L. reuteri strain, preferably as prepared according to the inventive method for pre-conditioning the probiotic L. reuteri strain, or as described herein, and an effective amount of GOS. A pre-conditioned probiotic L. reuteri strain is thereby a probiotic L. reuteri strain that has been cultivated in the presence of GOS, preferably according to the inventive method for pre-conditioning the probiotic L. reuteri strain. Administration allows preferably the prevention and/or treatment of a disease in a subject in need thereof or reducing or inhibiting the risk of such a disease. Any of the diseases as defined above are encompassed. The effective amount of a pre-conditioned probiotic L. reuteri strain and the effective amount of GOS may be provided in form of an inventive composition as described herein. Any of the features discussed above for the inventive method for pre-conditioning the probiotic L. reuteri strain and the inventive composition analogously apply.

The administration may involve the simultaneous administration of the probiotic composition comprising the effective amount of the pre-conditioned probiotic L. reuteri strain and the effective amount of GOS as a mixture or in a unit dosage. Alternatively, the administration may involve the simultaneous administration of the effective amount of the pre-conditioned probiotic L. reuteri strain and the effective amount of GOS but as provided as separate parts of a kit. In another embodiment, the administration involves separate administration of the effective amount of the pre-conditioned probiotic L. reuteri strain and the effective amount of GOS.

According to another particular embodiment, the current invention also provides the use of a pre-conditioned probiotic L. reuteri strain, preferably obtainable by the inventive method or otherwise as described herein for manufacturing or preparing a medicament or pharmaceutical composition for preventing and/or treating a disease in a subject in need thereof. Such a disease and a subject are preferably as described herein. The use preferably allows the prevention and/or treatment of a disease in a subject in need thereof or reducing or inhibiting the risk of such a disease. Any of the diseases as defined above are encompassed. Any of the features discussed above for the inventive method for pre-conditioning the probiotic L. reuteri strain and the inventive composition analogously apply.

Further aspects and embodiments are provided below.

A method for preparing a probiotic composition, characterized in that the method comprises the steps:

a) pre-conditioning of a probiotic Lactobacillus reuteri strain following the steps:

i) cultivating a probiotic L. reuteri strain in the presence of galacto-oligosaccharide (GOS) in a growth medium, thereby pre-conditioning the probiotic L. reuteri strain; and

ii) optionally harvesting the pre-conditioned probiotic L. reuteri strain from the growth medium; and

b) preparing a probiotic composition comprising the pre-conditioned probiotic L. reuteri strain obtained from step a)i) or a)ii) and adding GOS to the probiotic composition.

The method according to above, wherein GOS is added in step a)i) to the growth medium in an amount of at least 0.2 wt %, preferably at least 0.5 wt %, even more preferably at least 0.75 wt % of GOS in the growth medium, and preferably at most 8 wt % of GOS, such as at most 7 wt % GOS, at most 6 wt % GOS, or even at most 5 wt % GOS, more preferably at most 4 wt % of GOS or even at most 3% GOS, such as in a range of 0.2 wt % to 8 wt % or even 0.2 wt % to 7 wt %, or 0.2 wt % to 6 wt %, more preferably in a range of 0.5 wt % to 8 wt % or even 0.5 wt % to 7 wt %, or 0.5 wt % to 6 wt %, even more preferably in a range of 0.75 wt % to 8 wt % or even 0.75 wt % to 7 wt %, or 0.75 wt % to 6 wt %, and most preferably in a range of 0.75 wt % to 7 wt %, in a range of 0.75 wt % to 6 wt % or even in a range of 0.75 wt % to 5 wt %, such as a range of 0.75 wt % to 3 wt % or 0.75 wt % to 4 wt %, preferably calculated on basis of the growth medium (% w/w) preferably at the start, in the middle or at the end of cultivation of the probiotic L. reuteri strain in step a)i).

The method according to any above, wherein GOS is added in step b) to the pre-conditioned probiotic L. reuteri strain obtained from step a)i) or a)ii) in an amount of at least 1 or 2 g per daily dose, preferably at least 3 g per daily dose, likewise preferably between 2 to 12 g per daily dose, or between 2 to 10 g per daily dose, between 2 to 8 g per daily dose, or between 2 to 7 g per daily dose, more preferably between 3 to 12 g per daily dose, likewise more preferably between 3 and 10 g per daily dose, such as between 3 to 8 g per daily dose, between 3 to 7 g per daily dose, between 3 to 6 g per daily dose, or about 2, 3, 4, 5, 6, 7, 8, 9, 10, 11 or 12 g per daily dose or any range formed thereby.

The method according to any above, wherein GOS added to the growth medium in step a)i) and/or to the composition comprising the pre-conditioned probiotic L. reuteri strain prepared in step b) has a degree of polymerization (DP) of 3-8.

The method according to any above, wherein the probiotic L. reuteri strain is at least one L. reuteri strain or multiple L. reuteri strains selected from the group comprising or consisting of L. reuteri strain as deposited under DSM 17938, L. reuteri strain as deposited under ATCC PTA 5289, L. reuteri strain as deposited under ATCC PTA 6475, L. reuteri as deposited under DSM 32846, L. reuteri as deposited under DSM 32848, L. reuteri as deposited under DSM 32849, L. reuteri as deposited under DSM 27131, L. reuteri as deposited under DSM 32465 and L. reuteri strain as deposited under ATCC PTA 4659, preferably the probiotic L. reuteri strain is L. reuteri strain as deposited under DSM 17938.

A composition comprising:

an effective amount of a pre-conditioned probiotic Lactobacillus reuteri strain, wherein the pre-conditioned probiotic L. reuteri strain has been prepared by cultivating a L. reuteri strain in the presence of galacto-oligosaccharides (GOS) in a growth medium; and additionally

an effective amount of a GOS,

wherein the composition preferably has been prepared according to a method according to any above.

The composition according to above, wherein the composition comprises the pre-conditioned probiotic L. reuteri strain in an amount of between 10³ cfu to 10¹² cfu, typically in an amount of between 10⁴ cfu to 10¹¹ cfu per daily dose, preferably in an amount of between 10⁵ cfu to 10¹⁰ cfu per daily dose, or 10⁵ cfu to 10⁹ cfu per daily dose, likewise preferably in an amount of between 10⁶ cfu to 10⁹ cfu per daily dose, 10⁶ cfu to 10⁸ cfu per daily dose or in an amount of 10⁸ cfu to 10¹⁰ cfu per daily dose, more preferably around 10⁷ cfu to 10⁹ cfu per daily dose, most preferably in an amount of around 10⁸ total cfu per daily dose, such as 10⁷ to 10⁹ cfu per daily dose or 10⁸ to 10⁹ cfu per daily dose.

The composition according to any above, wherein the composition comprises GOS in an amount of at least 1 or 2 g per daily dose, preferably at least 3 g per daily dose, likewise preferably between 2 to 12 g per daily dose, or between 2 to 10 g per daily dose, between 2 to 8 g per daily dose, or between 2 to 7 g per daily dose, more preferably between 3 to 12 g per daily dose, likewise more preferably between 3 and 10 g per daily dose, such as between 3 to 8 g per daily dose, between 3 to 7 g per daily dose, between 3 to 6 g per daily dose, or about 2, 3, 4, 5, 6, 7, 8, 9, 10, 11, or 12 g per daily dose or any range formed thereby.

The composition according to any above, wherein

the composition is solid or liquid, and/or is present in form of a powder, a tablet, a capsule, or may be in form of an oil formulation, an emulsion, an oil-in-water emulsion (o/w emulsion), or a water-in oil emulsion (w/o emulsion); and/or

the composition is an administrable form, preferably selected from the group consisting of nutritional compositions, pharmaceutical formulations, dietary supplements, functional food products, functional beverage products, and combinations thereof, e.g., a dairy product, a milk-based product, a whey protein-based beverage.

Non-therapeutic use of a composition comprising an effective amount of a pre-conditioned probiotic Lactobacillus reuteri strain supplemented with an effective amount of galacto-oligosaccharides (GOS), the composition being prepared according to any above or is a composition according to any above, for improving or increasing the probiotic effect, preferably of a L. reuteri strain, in the digestive tract of a healthy subject.

The non-therapeutic use according to above, wherein improving or increasing the probiotic effect: is an increase of mineral bio-accessibility, preferably an increase of mineral solubility and/or mineral absorption, in the intestinal tract of the healthy subject, wherein the mineral is preferably selected from calcium and/or iron; and/or

is a promotion of a healthy microbiota in the intestinal tract of the healthy subject; and/or

is a promotion of the survival of the L. reuteri strain in the colon and thus a promotion of the health benefits associated to the L. reuteri strain; and/or

is an increase of metabolic activity of the gut microbiome such as an increase in the lactic acid/acetic acid production in the intestinal tract of the healthy subject; and/or

is an improvement of bone health, bone strength, bone formation, teeth conditions, teeth growth, muscular functions, cognitive functions, immune functions and/or general growth and development (especially for children/infants) due to an increase of mineral solubility and/or mineral absorption, in the intestinal tract of the healthy subject.

A composition comprising an effective amount of a pre-conditioned probiotic Lactobacillus reuteri strain and an effective amount of galacto-oligosaccharides (GOS) as prepared according to any above or according to any above for use as a medicament.

A composition comprising an effective amount of a pre-conditioned probiotic Lactobacillus reuteri strain and an effective amount of galacto-oligosaccharides (GOS) as prepared according to any above or according to any above for use in the prevention and/or treatment of mineral deficiency in a patient in need thereof, wherein the patient is preferably at risk of developing mineral deficiency or is a subject suffering from mineral deficiency, wherein the mineral is optionally selected from calcium and/or iron.

The composition for use according to above, wherein the composition is for use in the prevention and/or treatment of:

iron deficiency, preferably iron deficiency anemia, and/or promote cognitive functions, and/or promote oxygen transport, and/or promote immune functions, and/or reduces tiredness or fatigue, in a patient in need thereof; and/or calcium deficiency in a patient in need thereof.

The composition for use according to any above, wherein the composition is for use in the prevention and/or treatment of a digestive dysfunction, an impaired microbiota, and/or a digestive dysfunction.

A composition comprising an effective amount of a pre-conditioned probiotic Lactobacillus reuteri strain and an effective amount of galacto-oligosaccharides (GOS) as prepared according to any above or according to any above for use in the prevention and/or treatment of an impaired microbiota or a dysbiosis.

A composition comprising an effective amount of a pre-conditioned probiotic Lactobacillus reuteri strain and an effective amount of galacto-oligosaccharides (GOS) as prepared according to any above or according to any above for use in the prevention and/or treatment of bone loss and/or to promote growth and development of bones or teeth in children, and/or promote muscular functions, and/or promote functions of the nervous system and/or bone or muscular related conditions in a patient in need thereof.

A composition comprising an effective amount of a pre-conditioned probiotic Lactobacillus reuteri strain and an effective amount of galacto-oligosaccharides (GOS) as prepared according to any above or according to any above for use in the prevention and/or treatment of infections, preferably gut infections, and/or to promote immune functions, optionally through the increase of the lactic acid/acetic acid production in the microbiota of the patient.

It should be appreciated that the various aspects and embodiments of the detailed description as disclosed herein are illustrative of the specific ways to make and use the invention and do not limit the scope of invention when taken into consideration with the claims and the detailed description. It will also be appreciated that features from aspects and embodiments of the invention may be combined with further features from the same or different aspects and embodiments of the invention.

As used in this detailed description and the appended claims, the singular forms “a,” “an” and “the” include plural referents unless the context clearly dictates otherwise.

All ranges described are intended to include all numbers, whole or fractions, contained within the said range.

### EXAMPLES

### Example 1—Method for Pre-Conditioning L. reuteri with GOS

Internally produced L. reuteri frozen starter cultures (L. reuteri DSM 17938) were used to inoculate a fermentation medium containing 4% Bovine milk derived oligosaccharides (BMOS) as carbon source and 4% fructose as electron acceptor (on dry matter). Overnight culture of L. reuteri was used to inoculate a fresh fermentation medium containing 6% BMOS as carbon source and 6% fructose as electron acceptor (on dry matter). Fermentation was run under non pH control and at 37° C. Pre-conditioned L. reuteri cells were harvested by centrifugation after around 10 h fermentation prior to mixing with protectants and to spray-drying.

BMOS carbohydrate mixture used as source of GOS was composed of 48% GOS, 30% lactose, 8% glucose, 3.9% galactose, and >0.2% 3′-SL+6′-SL (g/100 g of dry matter).

L. reuteri cell counts were determined by pour plating. Briefly, serial decimal dilutions spray dried samples were performed in tryptone salt (Oxoid, LP0042) solution. Subsequently, 100 μL of the appropriate dilutions were transferred to Petri dishes and mixed with MRS agar (AES Chemunex, Bruz, France). Analysis was performed in duplicate. Plates were then incubated in aerobic conditions at 37° C. for 48 h. Colony forming units per gram (cfu/g) were calculated from the number of colonies counted on plates with appropriate dilution by determining their arithmetic mean.

Furthermore, according to an optional step, highly purified GOS (>93%) was added to the harvested pre-conditioned probiotic L. reuteri.

### Example 2—Manufacture of a Sachet Comprising Pre-Conditioned L. reuteri and GOS

The composition is made of: lyophilized pre-conditioned L. reuteri DSM 17938 (cultivated and lyophilized as described below), 10⁸ CFU/sachet and GOS 15 (VIVINAL®, FrieslandCampina Domo,

The Netherlands) 2000 mg/sachet. The composition is filled at ambient temperature into aluminium foil bags as known in the art with desiccant (10 cm×12 cm, using packaging material PET12/PE/ALU12/PE/PE+desiccant/PE from Alcan) in a LAF bench (Holten Laminair Model S-2010 1.2 from Heto-Holten A/S, Denmark). To each bag, 2 g of powder with L. reuteri and GOS is added using the balance XP-600 from Denver Instrument GmbH, Germany. The filled aluminum foil bags are then heat sealed with the film sealing device mode F460/2 from Kettenbaum Folienschweisstechnik GmbH & Co. KG, Germany.

### Cultivation in Bioreactor

Ten ml MRS media (Oxoid) is inoculated with 100 μl of L. reuteri DSM 17938 from a frozen stock stored at −70° C. The tube is incubated for 6 h at 37° C. and thereafter 500 μl is used for inoculation of a tube with 50 ml MRS. The tube is incubated at 37° C. for 16-20 hours and thereafter the whole volume is used as inoculum to the bioreactor.

### Conditions for Cultivation in Bioreactor

Working volume: 1 L

Medium: industrial MRS (iMRS) with 3% BMOS+3% fructose

Aeration: No aeration

Temperature: 37° C.

Stirring: 250 rpm

pH: 6.00+/−0.1

Cultivation time: 6-10 h

### iMRS Ingredients

| Component | Quantity (g) |
|---|---|
| Yeast extract | 10 |
| Yeast peptone | 10 |
| Sodium citrate | 2 |
| Sodium acetate | 5 |
| K₂HPO₄ | 2 |
| MgSO₄ heptahydrate | 0.1 |
| Manganese sulfate heptahydrate | 0.05 |
| Tween 80 | 1 |
| L-cysteine | 0.1 |
| Purified water (800 ml) | |

Set pH to 6.5 with NaOH, autoclave (121° C. 15 min) and thereafter add sterile sugar solution (final volume 1000 ml). After cultivation, the bacteria are pelleted by centrifugation and suspended in 50 ml 10% sucrose.

### Lyophilization

The concentrated cell suspensions are dispensed into freeze-drying glass vials (1 ml/vial) and the vials are frozen in an ultra-freezer at −50° C. for 2 h before being transferred to a Labconco FreeZone Stoppering Tray Freeze-Dryer. The freeze-drying scheme is set-up as follows: Freezing step: −40° C. for 4 h; Main drying: −35° C. for 15 min at 0.102 mbar, −10° C. for 12.5 hat 0.102 mbar; secondary drying: −10° C. for 30 min at 0.01 mbar, 25° C. for 12 h at 0.01 mbar. At the end of the process the vials were capped under vacuum and stored at −20° C. until further use.

### Example 3—Method for Preparing a Probiotic Composition Comprising Pre-Conditioned L. reuteri Supplemented With Additional GOS

A cow milk-based toddler beverage, also called growing-up-milk, containing minerals adapted for the age group was prepared by adding an amount of 5×10⁶ cfu per daily dose pre-conditioned probiotic L. reuteri and an amount of GOS of 0.5 to 12 g per daily dose reconstituted growing-up-milk composition.

### Example 4—Composition Comprising Pre-Conditioned L. reuteri Supplemented With Additional GOS Increases Survival in Colon and Increases Calcium and Iron Solubility in Colon SHIME®-Model

In this experiment, a simplified simulation of the continuous Simulator of the Human Microbial Ecosystem (SHIME®) was used. This SHIME® model has been extensively used for more than 20 years for both scientific and industrial projects and has been validated with in vivo parameters (see for example Van den Abbeele et al., Arabinoxylo-Oligosaccharides and Inulin Impact Inter-Individual Variation on Microbial Metabolism and Composition, Which Immunomodulates Human cells, J. Agric. Food chem. 2018, 66(5): 1121-1130).

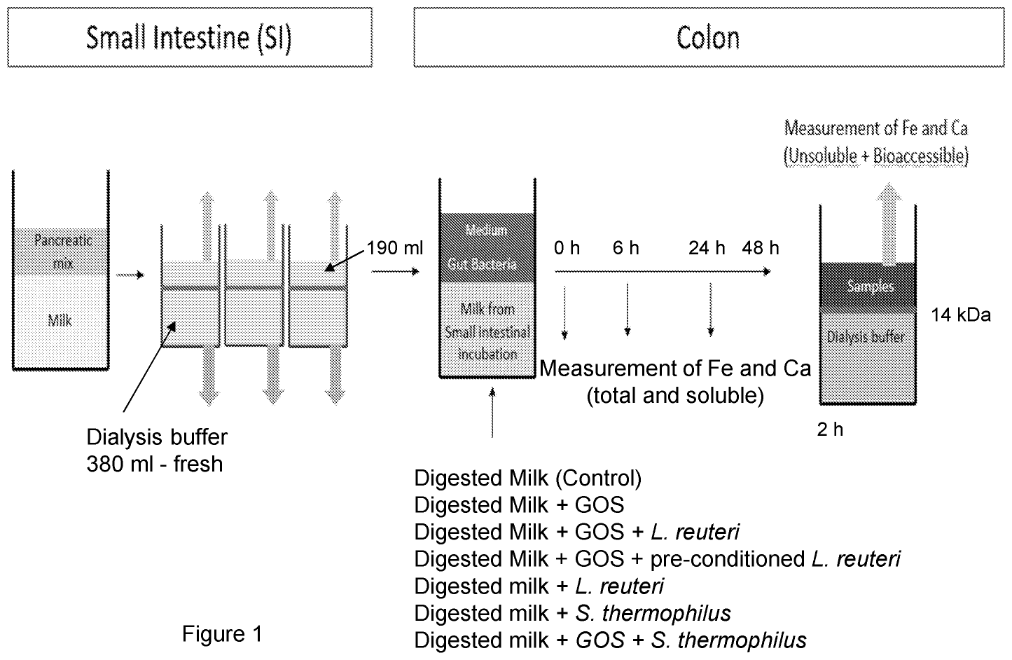

For the current set of experiments a two-stage batch system mimicking the upper gastro-intestinal tract (Upper GIT, stomach and small intestine) and colonic conditions were used as simplified SHIME® system (FIG. 1). Cow milk-based toddler beverage, also called Growing-up-milk, containing minerals adapted for the age group was used in these studies.

In order to simulate the absorptive processes occurring in the small intestine of toddlers, a dialysis approach was applied by using a cellulose membrane with a cut-off of 14 kDa. By introducing the small intestinal suspension within a dialysis membrane, molecules, such as digested amino acids, sugars, micronutrients and minerals, were gradually removed from the upper gastro-intestinal matrices.

Furthermore, a gradual pH decrease during the stomach incubation going from 5.5 till 3.0 during 1 h of incubation was implemented to simulate the gastric pH of toddlers. Also, during the first 30 minutes of small intestinal incubation (duodenum), a fixed pH of 4.5 was implemented to allow the available minerals to optimally absorb. The following 145 minutes of the small intestinal phase (jejunum+ileum), a pH of 7 was introduced. The milk matrix after exposure to gastric and small intestinal conditions was transferred to the colonic compartment containing the fecal sample of a toddler.

Fresh fecal material was collected from a 12-month-old infant donor. Fecal suspension was prepared and mixed with a protectant. At the start of the short-term colonic incubation, the test ingredients (L. reuteri strain and Streptococcus thermophilus strain) were added to sugar-depleted nutritional medium containing basal nutrients present in the colon (e.g., host-derived glycans such as mucin).

L. reuteri strain and S. thermophilus strain were inoculated at 1×10⁹ CFU/reactor.

Following incubations were performed:
Control incubation without probiotic (Upper GIT-digested milk alone)
Upper GIT digested milk+L. reuteri strain DSM 17938
Upper GIT digested milk+S. thermophilus NCC2496 (culture collection of Nestle S.A. (Vevey, Switzerland))
Upper GIT digested milk+GOS+L. reuteri strain DSM 17938
Upper GIT digested milk+GOS+S. thermophilus NCC2496

Highly purified GOS (>93% purity) was used at equivalent of 4 g GOS/L of digested milk to simulate GOS structures reaching the large intestine.

Internally produced S. thermophilus frozen starter cultures were used to inoculate a fermentation medium containing 3% dextrose as carbon source, 2.5% yeast extract as nitrogen source and 0.1% Tween® 80. Overnight culture at 37° C. of S. thermophilus was used to inoculate a fresh fermentation medium containing 4% dextrose as carbon source, 2.5% yeast extract as nitrogen source and 0.1% Tween® 80. Fermentation was run at 40° C. and under pH control maintained at 5.8 with addition of NaOH 30%.

In a separate experiment, the test ingredients (L. reuteri, pre-conditioned L. reuteri) were added into the colonic medium containing the fecal sample. BMOS was replaced by dextrose (8%) in the fermentation medium of L. reuteri (reference=not pre-conditioned). Same fermentation conditions were then applied (fermentation time of 10 h, no pH control, 37° C.).

L. reuteri strain and pre-conditioned L. reuteri strain were inoculated at 1x10 9 CFU/reactor. L. reuteri were quantified by plating on MRS medium, while S. thermophilus was plated on M17 medium supplemented with lactose. Both strains were incubated at 37° C. for 48h before cell counting. Quantities of L. reuteri and S. thermophilus powders were then calculated to target 1x10 9 CFU/colonic reactor.

Following incubations were performed:
Control incubation without probiotic (Digested milk alone)
Upper GIT digested milk+GOS
Upper GIT digested milk+L. reuteri strain DSM 17938
Upper GIT digested milk+pre-conditioned L. reuteri DSM 17938
Upper GIT digested milk+GOS+L. reuteri strain DSM 17938
Upper GIT digested milk+GOS+pre-conditioned L. reuteri DSM 17938

Colonic incubations were performed during 48 h, at 37° C. and under shaking (90 rpm) conditions. All experiments were performed in triplicate to account for biological variation.

### Analysis Of Minerals (Calcium and Iron)

Analysis of calcium and iron was performed by a specialized laboratory of Ghent University, Belgium. The samples were measured by inductively coupled plasma-optical emission spectrometry (ICP-OES). Samples for mineral analysis in the colon compartment were taken after 0 h, 6 h, 24 h and 48 h. The 48 h time point was preceded by a colonic dialysis, resulting in values for the bio-accessible mineral fraction and the insoluble mineral fraction. Samples taken at 0 h, 6 h and 24 h were centrifuged and filtered before analysis. Total calcium was measured before centrifugation. Soluble calcium was measured in the fraction after centrifugation and filtration (pellet excluded).

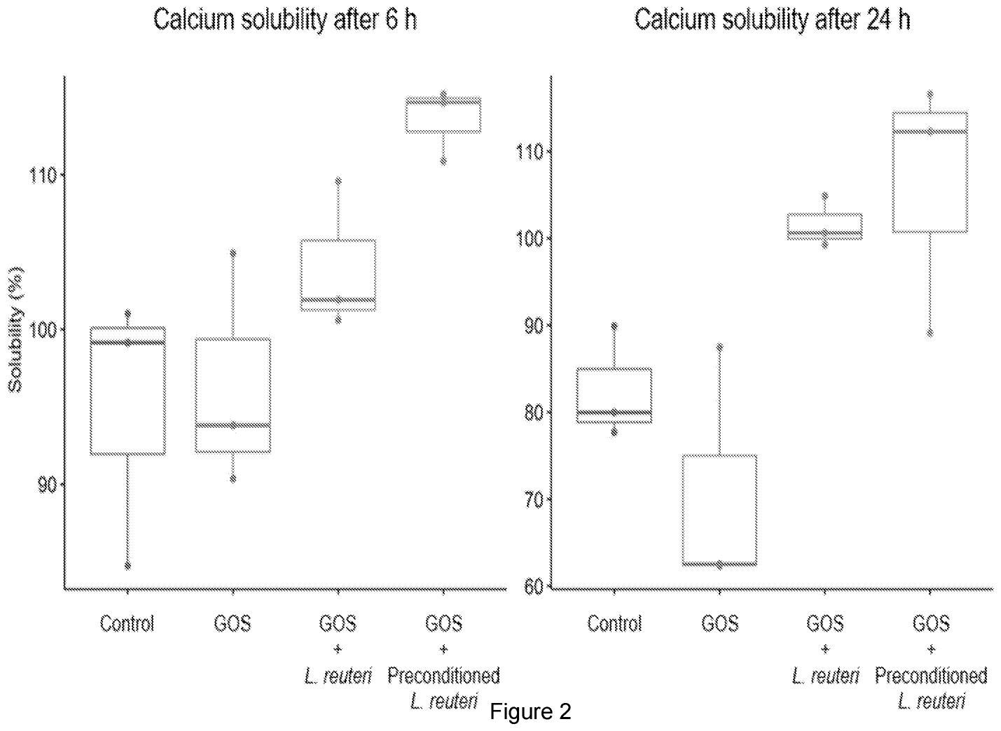

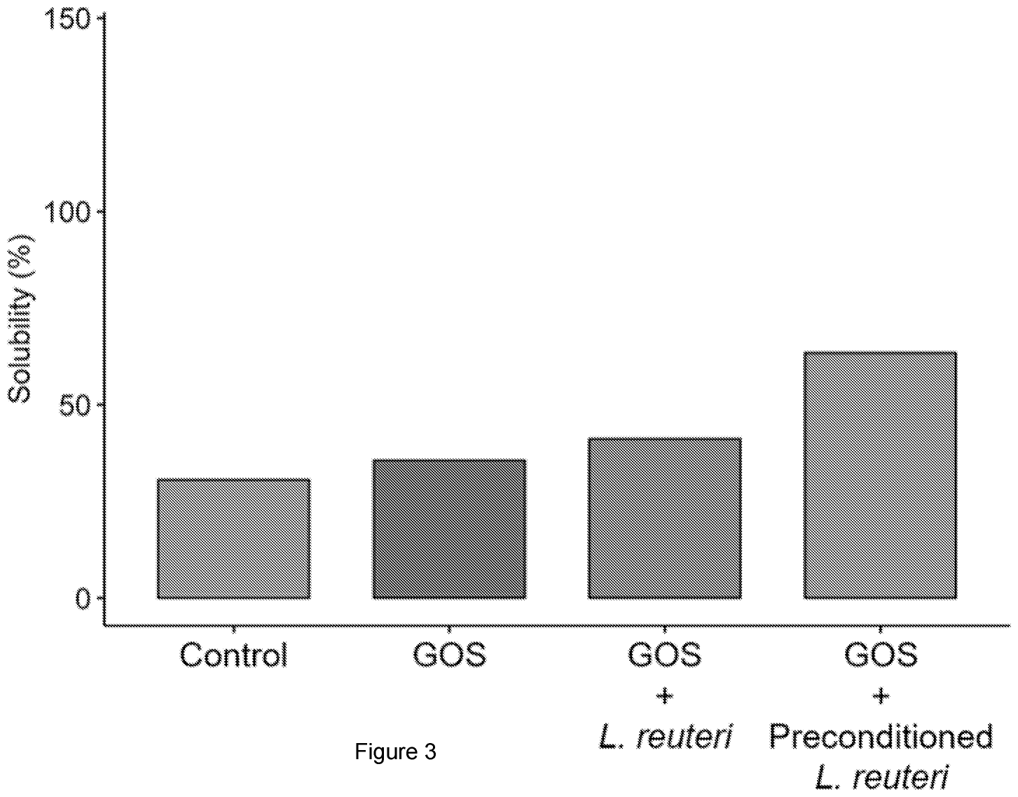

Results are shown in FIGS. 2 and 3.

As can be seen in FIG. 2, GOS alone has little or no effect on calcium solubility. L. reuteri combined with GOS boost the solubility of calcium at both 6 h and 24 h. Pre-conditioned probiotic L. reuteri strain further boosts calcium solubility in colon at both 6 h and 24 h in presence of GOS. This suggests that a pre-conditioned L. reuteri strain combined with GOS can be used to promote calcium absorption in a subject in need thereof.

As can be seen in FIG. 3, pre-conditioned probiotic L. reuteri strain in combination with GOS boosts iron solubility (bio-accessibility) in the colon suggesting that the combination of both GOS and pre-conditioned L. reuteri can promote iron absorption in a subject in need thereof.

### Microbial Community Composition

Quantitative polymerase chain reaction (qPCR) was performed to selectively enumerate Lactobacilli and Streptococci. qPCR was performed at 0 h and after 48 h of colonic incubation.

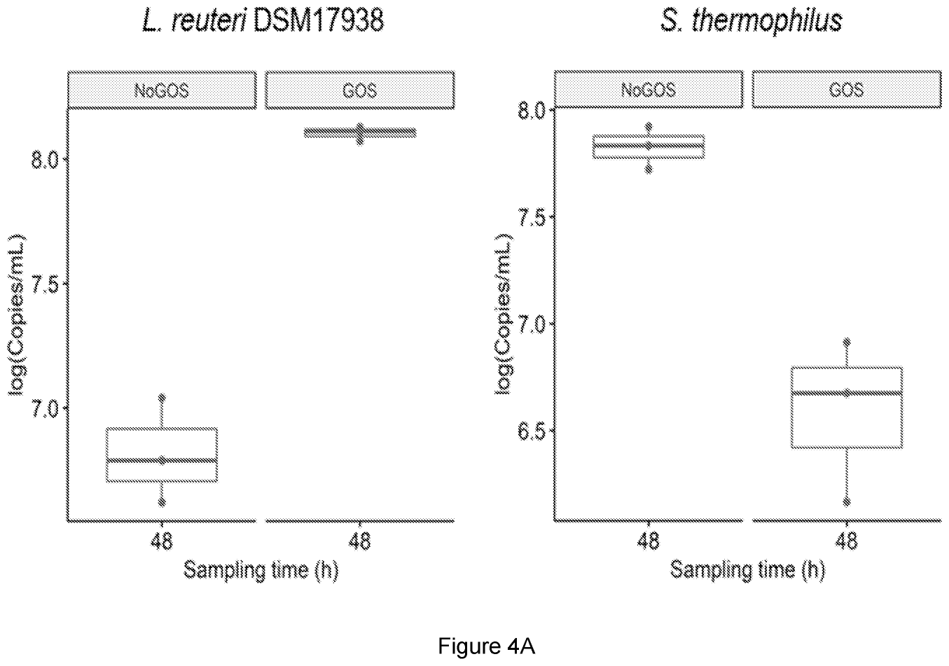

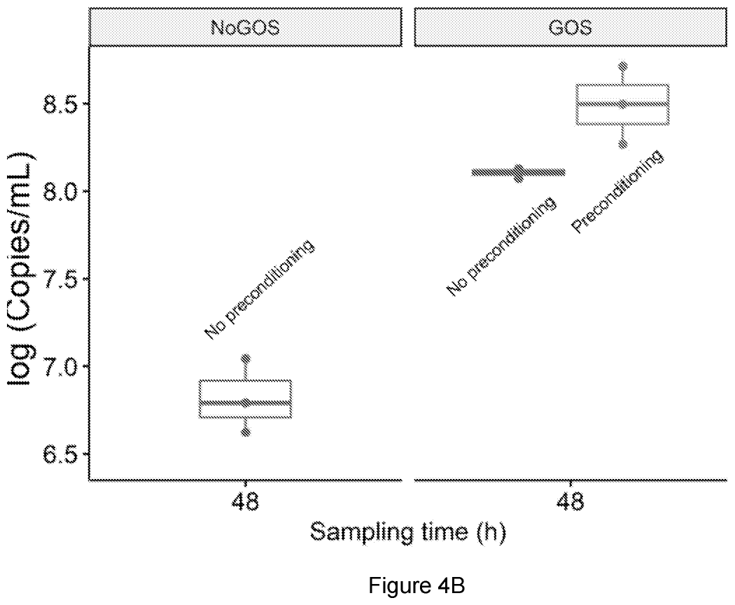

Results are shown in FIGS. 4A and 4B.

FIG. 4A shows that survival of L. reuteri in the colon is enhanced by the presence of GOS. In contrast, survival of S. thermophilus is impaired by the presence of GOS in the colon. It means that even if both strains are able to grow in presence of GOS in fermentation medium and under controlled conditions, the strains behave differently when they are in a highly competitive environment like in the colon. Diverse bacteria are present in the gut, especially in the colon. Bacteria fight for food to survive in the colonic environment. L. reuteri is able to consume GOS in an efficient manner leading to a good survival. S. thermophilus seems to struggle more in competitive bacterial environment even though the strain has the ability to consume GOS.

FIG. 4B shows the survival of pre-conditioned L. reuteri in the colon in presence of GOS. L. reuteri (no pre-conditioning) survives well in the colon in presence of GOS. Pre-conditioning improves the survival of L. reuteri as seen by higher cell counts at 48 h in the colon.

### Microbial Metabolic Activity

To assess the microbial activity of the pre-conditioned probiotic L. reuteri strain prepared according to the inventive method, the pH, short chain fatty acids and lactate values were monitored over a period of 0 h, 6 h and 48 h after starting the incubation:
pH: The degree of acidification during the experiment is a measure of the intensity of bacterial metabolism (fermentation). The pH of the incubations was determined at 0 h, 6 h and 48 h after starting the incubation, thus giving a rough indication on the speed of fermentation.
Short chain fatty acids (SOFA) are an assessment of the microbial carbohydrate metabolism (acetate in this case) can be compared to typical fermentation patterns for normal GI microbiota. Samples for SOFA analysis were analyzed after 0 h, 6 h and 48 h of incubation.
Lactate: The intestine harbors both lactate-producing and lactate-consuming bacteria. Lactate is produced by lactic acid bacteria and decreases the pH of the environment, acting also as an antimicrobial agent. It can also be rapidly converted to acetate, butyrate, and propionate by other microorganisms. Samples for lactate analysis were analyzed after 0 h, 6 h, and 48 h of incubation.

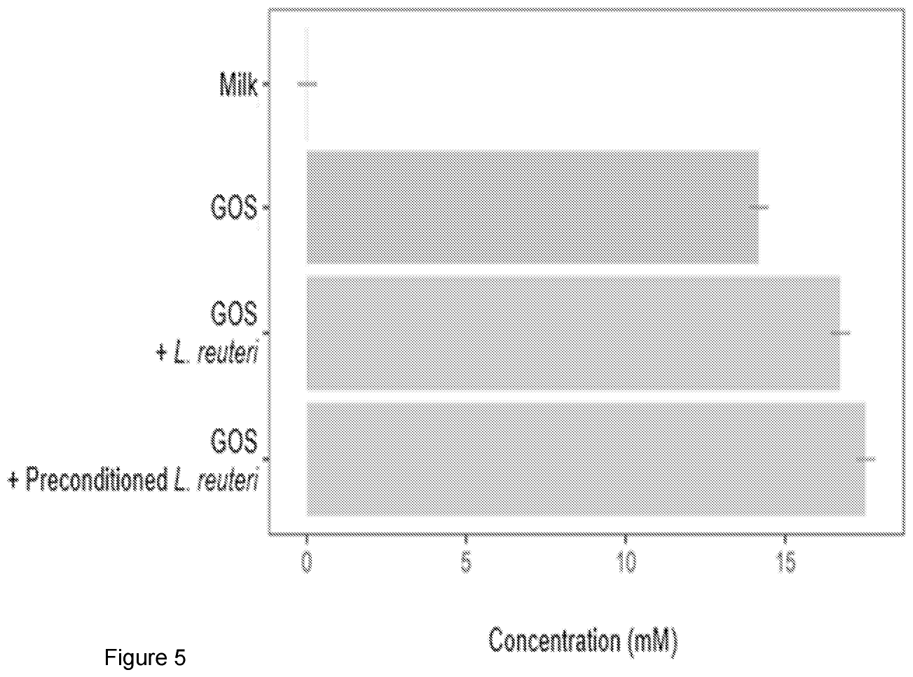

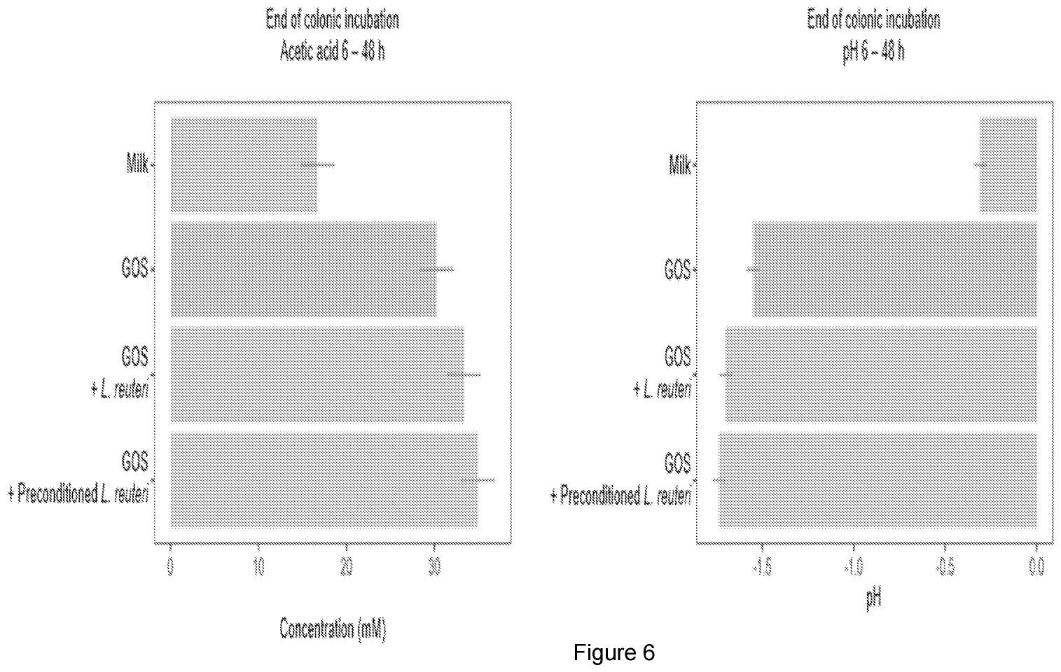

Results are shown in FIGS. 5 and 6.

FIG. 5 shows the lactic acid production (difference between the level at 48 h and 6 h). GOS alone induces a strong lactic acid production in the colon. Addition of L. reuteri in combination of GOS further enhances the production of lactic acid in the colon. The highest production of lactic acid was measured when pre-conditioned L. reuteri was added in combination of GOS in the colon, showing an overall increase of the metabolic activity of the microbiota. Lactic acid producing bacteria are more active in the colon when GOS and pre-conditioned L. reuteri are added for 48 h.

FIG. 6 shows the pH decrease in the colon (difference between 48 h and 6 h) (right panel) and acetic acid production (difference between 48 h and 6 h) (left panel). Pre-conditioned L. reuteri combined with GOS induces a stronger pH decrease in the colon. Higher level of acetic acid is also produced in presence of pre-conditioned L. reuteri and GOS. pH is a marker of acid production. Both lactic acid and acetic acid are strongly induced by pre-conditioned L. reuteri in presence of GOS.

### Example 5—Composition Comprising Pre-Conditioned L. reuteri Supplemented With Additional GOS Shows a Positive Effect on Bone Parameters

In vitro bone experiments were carried out using a murine pre-osteoblast cell model: MC3T3-E1 (osteoblast are the bone forming cells). Bone cells were exposed to different conditions (see below). These conditions were prepared after 48 h colonic incubations by centrifugation and filtration before being added at 1% to the bone cell cultures in DMEM medium.

### Conditions

Upper GI digested milk (control)
Upper GI digested milk+GOS (GOS)
Upper GI digested milk+GOS+L. reuteri strain (GOS+L. reuteri)
Upper GI digested milk+GOS+pre-conditioned L. reuteri strain (GOS+preconditioned L. reuteri)

### Read outs

Alkaline phosphatase (ALP) activity (enzymatic activity). The ALP is an enzyme expressed by osteoblasts cells when they mature and is an indicator of differentiation of osteoblasts (bone forming cells).
Alizarin red absorption is an indicator of osteoblast mineralization.
Scratch assay as indicator of MC3T3 cell migration

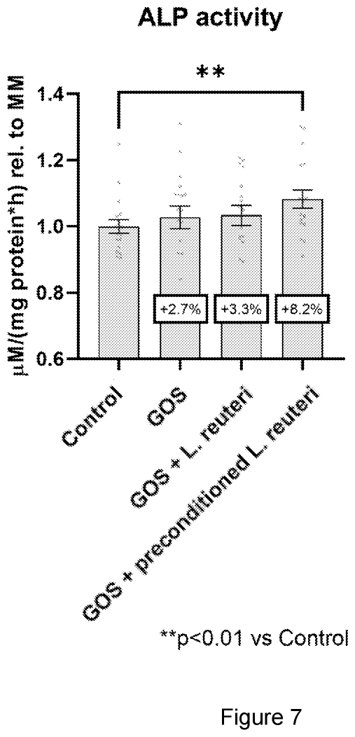

As can be seen in FIG. 7, a positive effect on osteoblast proliferation with a superiority of GOS+pre-conditioned L. reuteri strain compared to only GOS or GOS+L. reuteri strain has been observed. Impact on osteoblast differentiation is associated with a positive impact on bone formation rate thus speed of growth.

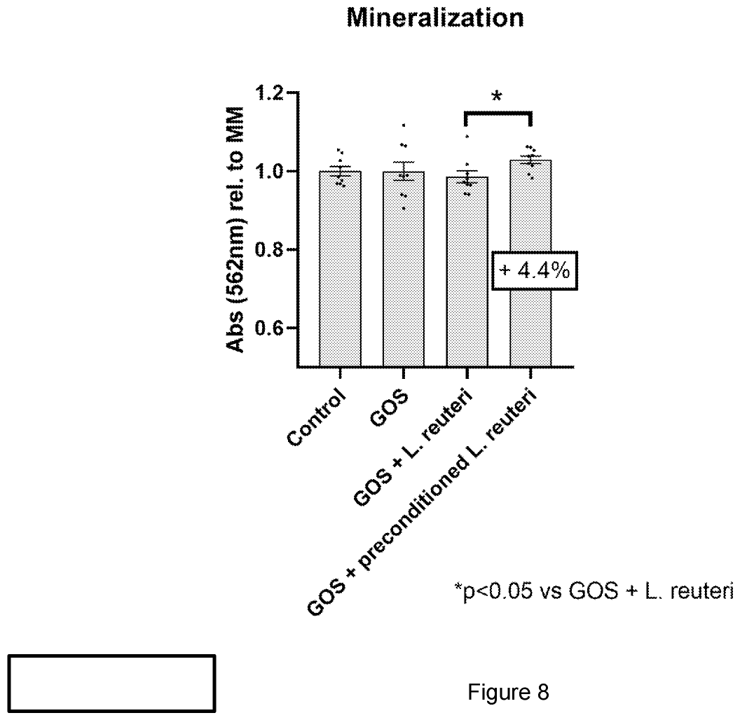

As shown in FIG. 8, mineralization (late osteoblast activity) is higher with GOS+pre-conditioned L. reuteri strain (vs GOS+L. reuteri strain). Higher mineralization may lead to increased bone strength.

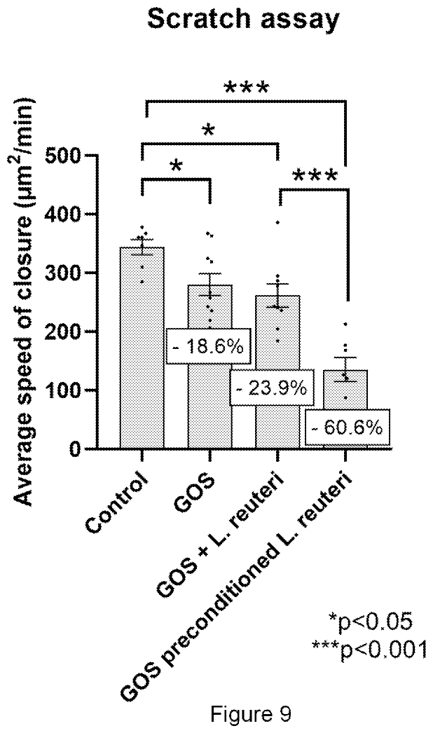

As shown in FIG. 9, GOS+pre-conditioned L. reuteri significantly decreased cell migration vs all the other conditions. This is consistent with an increased differentiation of cells when exposed to GOS+pre-conditioned L. reuteri, as shown in FIG. 7.

In vitro experiments were carried out using a murine pre-osteoblast cell model: MC3T3-E1 (osteoblast are the bone forming cells). Cells were exposed to different conditions:

Upper GIT-digested milk supplemented with GOS, with and without L. reuteri samples (with and without pre-conditioning) after 48 h colonic incubations were collected and levels of short-chain fatty acids (SCFAs), e.g., butyrate, propionate and acetate, were measured.

Cells were exposed to corresponding conditions in the table at 1/1000 for blends of pure SCFAs in DMEM. Conditions are listed in the table below.

| | Acetate | Propionate | Butyrate |
|---|---|---|---|
| Condition | (mM) | (mM) | (mM) |
| SCFAs (control) | 22.98 | 12.25 | 3.63 |
| SCFAs (GOS) | 46.05 | 14.20 | 3.85 |
| SCFAs (GOS + normal L. reuteri) | 51.89 | 13.42 | 3.78 |
| SCFAs (GOS + preconditioned | 56.60 | 17.41 | 4.56 |
| L. reuteri) | | | |

Gene expression read outs: Activating transcription factor 4 (Atf4) and Osteocalcin (Ocn) at days 4. Atf4 is an early marker of osteoblasts differentiation, Ocn is a marker of osteoblast mineralization.

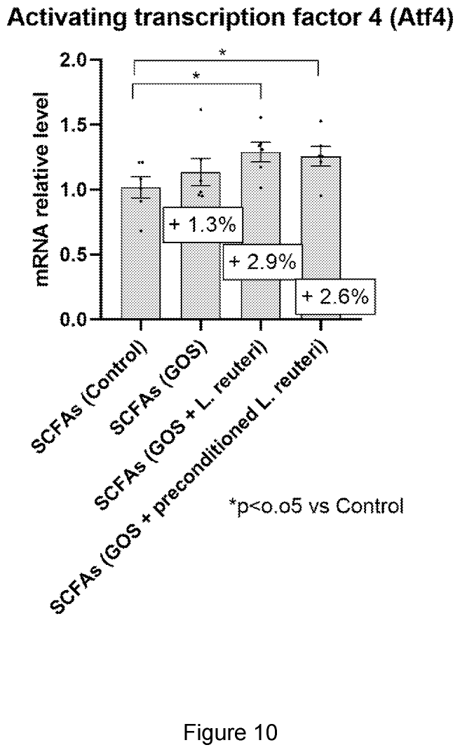

As shown in FIG. 10, both SCFAs blends from GOS+L. reuteri or GOS+pre-conditioned L. reuteri conditions increased significantly Atf4 gene expression vs control condition. This indicates a stimulation of early osteoblast differentiation.

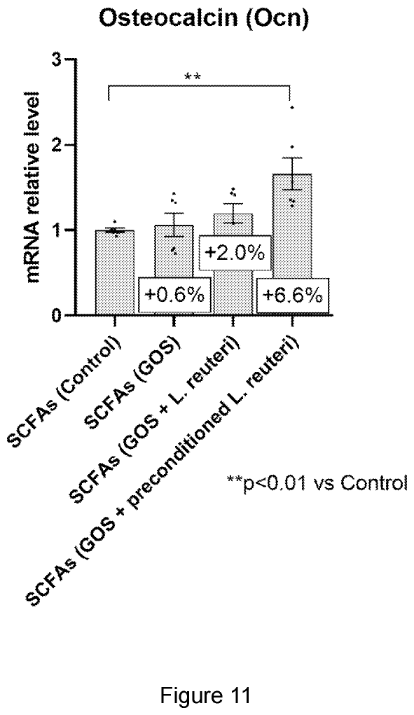

As shown in FIG. 11, only SCFAs blend from GOS+pre-conditioned L. reuteri conditions significantly increased Ocn gene expression vs control condition. This indicates a superiority of pre-conditioned L. reuteri to stimulate of osteoblast mineralization compared to GOS+L. reuteri (without pre-conditioning).

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method for preparing a probiotic composition, the method comprises the steps:
a) pre-conditioning of a probiotic Lactobacillus reuteri strain by:
ai) cultivating a probiotic L. reuteri strain in the presence of galacto-oligosaccharide (GOS) in a growth medium, thereby pre-conditioning the probiotic L. reuteri strain; and
aii) harvesting the pre-conditioned probiotic L. reuteri strain from the growth medium; and
b) adding GOS to the pre-conditioned probiotic L. reuteri strain obtained from step aii) to prepare a probiotic composition.

2-3. (canceled)

4. The method according to claim 1, wherein GOS is added in step ai) to the growth medium in an amount selected in a range of 0.2 wt % to 8 wt %.

5. (canceled)

6. The method according to claim 5, wherein step b) comprises adding GOS to the pre-conditioned probiotic L. reuteri strain obtained from step aii) in an amount of at least 1 g, or an amount selected from 2 to 12 g.

7. The method according to claim 1, wherein the GOS added to the growth medium in step ai) and/or to the pre-conditioned probiotic L. reuteri strain in step b) has a degree of polymerization (DP) of 3-8.

8. The method according to claim 1, wherein the probiotic L. reuteri strain is at least one L. reuteri strain selected from the group consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 33632, L. reuteri DSM 33633, L. reuteri DSM 33634, L. reuteri DSM 33635, L. reuteri DSM 27131, L. reuteri DSM 32465, L. reuteri DSM 32231, L. reuteri DSM 32232, L. reuteri ATCC PTA 6127 and L. reuteri ATCC PTA 4659.

9. The method according to claim 1, further comprising:
aiii) washing the pre-conditioned probiotic L. reuteri strain obtained from step aii) and/or
aiv) drying the pre-conditioned probiotic L. reuteri strain obtained from step aii), optionally wherein the drying comprises spray-drying or freeze drying, or the washed pre-conditioned probiotic L. reuteri strain obtained from step aiii).

10-11. (canceled)

12. A probiotic composition comprising:
an effective amount of a pre-conditioned probiotic Lactobacillus reuteri strain, wherein the pre-conditioned probiotic L. reuteri strain has been prepared by cultivating a L. reuteri strain in the presence of galacto-oligosaccharides (GOS) in a growth medium; and
an effective amount of GOS.

13. The probiotic composition according to claim 12, wherein the probiotic composition comprises the pre-conditioned probiotic L. reuteri strain in an amount of from 10³ cfu to 10¹² cfu.

14. (canceled)

15. The probiotic composition according to claim 12, wherein the effective amount of GOS in an amount of at least 1 g, or an amount selected from 2 to 12 g.

16. The probiotic composition according to claim 12, wherein the probiotic composition is in a form selected from the group consisting of a powder, a tablet, a capsule, an oil formulation, an emulsion, an oil-in-water emulsion (o/w emulsion), and a water-in oil emulsion (w/o emulsion).

17. The probiotic composition according to claim 12, wherein the probiotic composition is in an administrable form selected from the group consisting of a nutritional composition, a pharmaceutical formulation, a dietary supplement, a functional food product, and a functional beverage product.

18. The probiotic composition according to claim 12, wherein the L. reuteri strain is at least one L. reuteri strain selected from the group consisting of L. reuteri DSM 17938, L. reuteri ATCC PTA 5289, L. reuteri ATCC PTA 6475, L. reuteri DSM 32846, L. reuteri DSM 32847, L. reuteri DSM 32848, L. reuteri DSM 32849, L. reuteri DSM 33632, L. reuteri DSM 33633, L. reuteri DSM 33634, L. reuteri DSM 33635, L. reuteri DSM 27131, L. reuteri DSM 32465, L. reuteri DSM 32231, L. reuteri DSM 32232, L. reuteri ATCC PTA 6127 and L. reuteri ATCC PTA 4659.

19. The probiotic composition according to claim 12, comprising
an effective amount of a freeze dried pre-conditioned probiotic L. reuteri strain; and
the effective amount of GOS.

20. (canceled)

21. A method for improving or increasing the survival, persistence and/or probiotic effect of a L. reuteri strain in the digestive tract of a healthy subject, the method comprising administering to the healthy subject an effective amount of the probiotic composition of claim 12, wherein improving or increasing the probiotic effect:
is an increase of mineral bio-accessibility, optionally an increase of mineral solubility and/or mineral absorption, in the intestinal tract of the healthy subject; and/or
is a promotion of a healthy microbiota in the intestinal tract of the healthy subject; and/or
is a promotion of the survival of the L. reuteri strain in the colon and thus a promotion of the health benefits associated to the L. reuteri strain; and/or
is an increase of metabolic activity of the gut microbiota, optionally is an increase in the lactic acid/acetic acid production in the intestinal tract of the healthy subject; and/or
is an increase of the bone formation and/or bone mineralization and/or an increase of the maturation and/or activity of osteoblasts in the healthy subject.

22-23. (canceled)

24. A method for treating or reducing the risk of developing a mineral deficiency in a subject in need thereof, the method comprising administering to the subject an effective amount of the probiotic composition according to claim 12, optionally, wherein the mineral deficiency is a deficiency in calcium or iron.

25. (canceled)

26. The method of claim 24, wherein the subject in need thereof has iron deficiency anemia.

27. A method of treating or reducing the risk of developing a digestive dysfunction in a subject in need thereof, the method comprising administering to the subject an effective amount of the probiotic composition according to claim 12.

28. A method of treating or reducing the risk of developing a dysbiosis in a subject in need thereof, the method comprising administering to the subject an effective amount of the probiotic composition according to claim 12.

29. A method of treating or reducing the risk of developing bone loss in a subject in need thereof, the method comprising administering to the subject an effective amount of the probiotic composition according to claim 12.

30. A method for promoting growth and development of bones or teeth in a child, the method comprising administering to the child the composition of claim 12.

31. A method of treating or reducing the risk of developing and infection in a subject in need thereof, the method comprising administering to the subject an effective amount of the probiotic composition according to claim 12, optionally wherein the infection is a gut infection.
